(19) United States
(12) Patent Application Publication
(10) Pub. No.: US 2016/0074016 A1
(43) Pub. Date: Mar. 17, 2016

(54) TRANSMIT BEAMFORMING APPARATUS, RECEIVE BEAMFORMING APPARATUS, ULTRASONIC PROBE HAVING THE SAME, AND BEAMFORMING METHOD

(71) Applicant: SAMSUNG ELECTRONICS CO., LTD., Suwon-si (KR)

(72) Inventors: Suhyun PARK, Hwaseong-si (KR); Kyuhong KIM, Seoul (KR); Baehyung KIM, Yongin-si (KR); Jong Keun SONG, Yongin-si (KR); Seungheun LEE, Seongnam-si (KR); Taeho JEON, Seoul (KR); Kyungil CHO, Seoul (KR)

(73) Assignee: SAMSUNG ELECTRONICS CO., LTD., Suwon-si (KR)

(21) Appl. No.: 14/837,622

(22) Filed: Aug. 27, 2015

(30) Foreign Application Priority Data
Sep. 11, 2014 (KR) 10-2014-0120336

(51) Int. Cl. A61B 8/00

(52) U.S. Cl. CPC A61B 8/54; A61B 8/467; A61B 8/461

(57) ABSTRACT

A transmit beamforming apparatus, receive beamforming apparatus, ultrasonic probe having the same, ultrasonic diagnostic apparatus, and beamforming method are provided. The transmit beamforming apparatus for transmitting ultrasound beams by using a plurality of ultrasonic transducer elements includes a transmit beamformer configured for forming a transmit signal pattern by applying a delay time to a transmit signal that corresponds to at least one of the plurality of ultrasonic transducer elements; and a transmission controller configured for determining a delay frequency to be applied in conjunction with the application of the delay time.

### CROSS-REFERENCE TO RELATED APPLICATION

This application claims priority from Korean Patent Application No. 10-2014-0120336, filed on Sep. 11, 2014, in the Korean Intellectual Property Office, the disclosure of which is incorporated hereby incorporated by reference in its entirety.

### TECHNICAL FIELD

Apparatuses and methods consistent with exemplary embodiments relate to beamforming in an ultrasonic diagnostic apparatus.

### BACKGROUND

Ultrasonic diagnostic equipment irradiates ultrasonic signals from the surface of a subject toward a target region inside the body, and noninvasively obtains a cross-sectional tomogram of a soft tissue or an image of the blood flow using a reflected ultrasonic signal (or ultrasonic echo signal).

The ultrasonic diagnostic equipment is widely used in diagnosing diseases in the heart, abdominal region, urinary organs, and organs unique to women, because it is small, inexpensive, able to display real-time images, and safe without being exposed to radiation like X-rays, as compared to other diagnostic imaging equipment, such as X-ray imaging devices, Magnetic Resonance Imaging (MRI) devices, nuclear medicine diagnostic devices, etc.

Transducer arrays for converting between electric signals and ultrasound signals are used to irradiate ultrasonic signals to the inside of the body, and each includes a plurality of transducer elements. To focus the ultrasound signals generated by the plurality of transducer elements to a spot inside the body at the same time or to overcome a difference in time of ultrasound echo signals reflecting back from the spot and arriving at the respective transducer elements, an appropriate time delay is applied to the irradiated ultrasound signals or the received ultrasound echo signals, which is called “beamforming.”

Beamforming may be divided into analog beamforming and digital beamforming. Analog beamforming has an advantage of helping to enable the hardware to be miniaturized, because analog-to-digital converters (ADCs) are not required for the respective channels in the analog beamforming scheme.

### SUMMARY

One or more exemplary embodiments provide a transmit beamforming apparatus, receive beamforming apparatus, ultrasonic probe having the same, and beamforming method, by which complexity of hardware design of an analog beamforming circuit and an amount of data to be used to control beamforming may be reduced by dynamically setting the delay resolution.

In accordance with an aspect of an exemplary embodiment, a transmit beamforming apparatus for transmitting ultrasound beams is provided. The transmit beamforming apparatus includes a transmit beamformer configured to form a transmit signal pattern by applying a delay time to a transmit signal that corresponds to at least one from among a plurality of ultrasonic transducer elements; and a transmission controller configured to determine a delay frequency to be applied to the transmit signal in conjunction with the application of the delay time.

The transmit beamforming apparatus may further include a clock generator configured to generate a transmit clock signal which is usable for controlling an output timing of the transmit signal.

The clock generator may be synchronized to the delay frequency to generate the transmit clock signal.

The transmission controller may be further configured to determine the delay frequency based on at least one from among whether the plurality of ultrasonic transducer elements is arranged in a two-dimensional (2D) array, a focal depth, a number of ultrasonic transducer elements to be activated, a steering angle, and an aperture size.

The transmission controller may be further configured to determine the delay frequency to vary inversely with respect to the focal depth.

The transmission controller may be further configured to determine the delay frequency to vary inversely with respect to the number of the ultrasonic transducer elements to be activated.

The transmission controller may be further configured to determine the delay frequency to vary inversely with respect to the aperture size.

The transmission controller may be further configured to determine the delay frequency to be lower when the plurality of ultrasonic transducer elements is arranged in a 2D array than when the plurality of ultrasonic transducer elements is arranged in a 1D array.

The transmission controller may be further configured to calculate a first delay time for the at least one from among the plurality of ultrasonic transducer elements based on at least one from among whether the plurality of ultrasonic transducer elements is arranged in a two-dimensional (2D) array, the focal depth, the number of ultrasonic transducer elements to be activated, the steering angle, and the aperture size.

The transmission controller may be further configured to calculate a second delay time for the at least one from among the plurality of ultrasonic transducer elements based on the determined delay frequency and the first delay time.

The transmit beamformer may be further configured to form a transmit signal pattern by applying the second delay time to transmit signals that correspond to the at least one from among the plurality of ultrasonic transducer elements.

In accordance with an aspect an exemplary embodiment, a receive beamforming apparatus for focusing of analog receive signals is provided, wherein the analog receive signals are converted from ultrasound echo signals received by at least one from among a plurality of ultrasonic transducer elements. The receive beamforming apparatus includes a receive beamformer configured to apply a delay time to the analog receive signals converted by at least one from among the plurality of ultrasonic transducer elements and to focus the delayed analog receive signals; and a reception controller configured to determine a delay frequency to be applied to the analog receive signals in conjunction with the application of the delay time.

The receive beamformer may be further configured to hold the analog receive signals in order to apply the delay time to the analog receive signals.

The receive beamforming apparatus may further include a clock generator configured to generate a receive clock signal which is usable for controlling a holding time for the analog receive signal to be held by the receive beamformer.

The clock generator may be synchronized to the delay frequency in order to generate the receive clock signal.

The receive beamformer may be further configured to sample and hold the analog receive signals received from the at least one ultrasonic transducer element based on the applied delay time, and to simultaneously output the sampled and held analog receive signals.

Sampling timing of the receive beamformer may be controlled by the receive clock signal.

Other aspects, advantages, and salient features of the exemplary embodiments will become apparent to those of skill in the art from the following detailed description, which, taken in conjunction with the annexed drawings, discloses exemplary embodiments of the present disclosure.

### BRIEF DESCRIPTION OF THE DRAWINGS

The above and/or other aspects will become more apparent by describing certain exemplary embodiments with reference to the accompanying drawings, in which:

FIG. 1 illustrates transmit beamforming with one dimensional (1D) array transducers;

FIG. 2 illustrates delays of transmit signals to be applied in transmit beamforming;

FIG. 3 is a control block diagram of a transmit beamforming apparatus, according to an exemplary embodiment;

FIG. 4 schematically illustrates patterns of transmit signals output from a transmit beamformer and signal flows of the transmit signals until being focused on a focal point;

FIG. 5 illustrates a clock frequency of a clock generator and clock signals input to a transmit beamformer;

FIG. 6 is a graph representing beam profiles obtained at different clock frequencies with respect to a 64×1 1D transducer array;

FIGS. 7 and 8 show a change in beamforming effects, which appears when delay information grows in a 1D transducer array;

FIG. 9 is a graph representing beam profiles of a 64×1 1D transducer array;

FIG. 10 is a graph representing beam profiles of a 64×16 2D transducer array;

FIG. 11 is a graph representing beam profiles of a 64×64 2D transducer array;

FIG. 12 shows a difference in delay time that varies as a function of focal depth;

FIG. 13 is a graph representing beam profiles of a 64×64 2D transducer array at 10 mm of focal depth;

FIG. 14 shows a situation in which activated transducer elements remain the same even when a focal point is shifted laterally;

FIG. 15 shows a situation in which activated transducer elements are changed when a focal point is shifted laterally;

FIG. 16 is a control block diagram of an ultrasonic probe to implement the situation of FIG. 15;

FIG. 17 shows changes in aperture size in case the focal point is shifted to the axial direction;

FIG. 18 shows a diagram which illustrates how a transmission controller calculates delay time for a transducer element;

FIG. 19 is a graph representing beam profiles with a focal distance being 5 mm and 16×16 2D array elements being activated;

FIG. 20 is a graph representing beam profiles with a focal distance being 30 mm and 16×16 2D array elements being activated;

FIG. 21 shows a delay of receive signals to be applied in receive beamforming;

FIG. 22 shows dynamic reception focusing to be applied in receive beamforming;

FIG. 23 is a control block diagram of a receive beamforming apparatus, according to an exemplary embodiment;

FIG. 24 shows patterns of receive clock signals input to a receive beamformer and receive signals output from the receive beamformer, in case of performing receive beamforming according to a sample and hold scheme;

FIG. 25 is a timing diagram of receive clock signals for respective elements;

FIG. 26 is a control block diagram of an ultrasonic probe, according to an exemplary embodiment;

FIG. 27 is an exterior view of an ultrasonic probe that includes 1D array transducers;

FIG. 28 is an exterior view of an ultrasonic probe that includes 2D array transducers;

FIG. 29 is a control block diagram of transmit and receive beamforming apparatuses that share a clock generator and a controller;

FIG. 30 is a control block diagram of an ultrasonic diagnostic apparatus, according to an exemplary embodiment;

FIG. 31 is an exterior view of an ultrasonic diagnostic apparatus, according to an exemplary embodiment;

FIG. 32 is a flowchart illustrating a transmit beamforming method, according to an exemplary embodiment; and

FIG. 33 is a flowchart illustrating a receive beamforming method, according to an exemplary embodiment.

Throughout the drawings, like reference numerals will be understood to refer to like parts, components, and structures.

### DETAILED DESCRIPTION

Exemplary embodiments will be described in detail with reference to accompanying drawings.

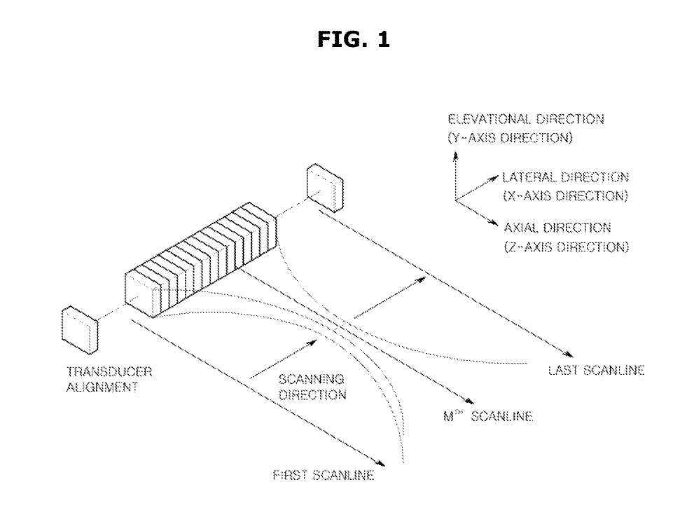

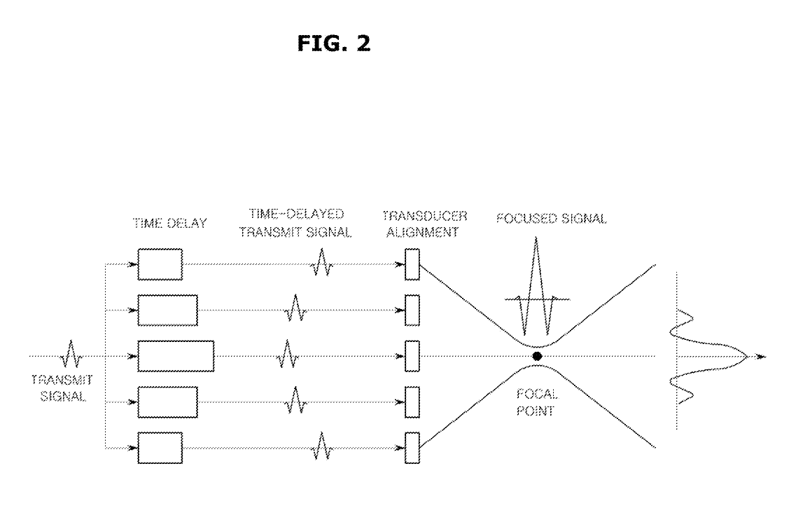

FIG. 1 illustrates transmit beamforming with one dimensional (1D) array transducers, and FIG. 2 illustrates delays of transmit signals to be applied in transmit beamforming.

Referring to FIG. 1, a three dimensional (3D) space subject to ultrasonic imaging may be defined by the X-axis corresponding to a lateral direction, the Y-axis corresponding to an elevational direction, and the Z-axis corresponding to an axial direction.

Spatial resolution of a two dimensional (2D) ultrasonic image may be determined by axial and lateral resolution. Axial resolution refers to the ability to distinguish two objects that lie along the axis of the ultrasound beam, and lateral resolution refers to an ability to distinguish two objects that lie on a line perpendicular to the axis of the ultrasound beam.

Axial resolution is determined by the pulse width of the transmit ultrasound signal, and a high frequency ultrasound signal with a short pulse width yields better axial resolution. Since the lateral resolution and elevational resolution are determined by the width of the ultrasound beam, the narrower the width of the ultrasound beam, the better the lateral resolution.

Accordingly, to improve the resolution of an ultrasound image, especially the lateral resolution, an ultrasound beam having a narrow beamwidth may be formed by focusing ultrasound signals transmitted from a plurality of transducer elements to a focal point on the scan line, which is called transmit beamforming.

The 1D transducer array is comprised of a plurality of transducer elements arrayed in a one-dimensional arrangement. To obtain 2D ultrasound cross sectional images, a plurality of scan lines are required, and aforementioned beamforming on the focal point may be performed from the first scan line to the last scan line.

A 2D ultrasound cross sectional image on the X-Y plane may be obtained by transmitting ultrasound signals to all of the scan lines and receiving the ultrasound echo signals which are reflected from the internal substances of a subject.

To focus ultrasound beams on a particular spot, the ultrasound signals transmitted from the plurality of transducer elements are to be controlled to arrive simultaneously at the particular spot, i.e., a focal point. As shown in FIG. 2, since distances from the respective transducer elements to the focal point are different, appropriate time delays are to be applied to the respective ultrasound signals transmitted from the corresponding transducer elements (hereinafter, referred to as ‘elements’) such that the ultrasound signals may reach the focal point at the same time.

Referring to FIG. 2, assuming that the ultrasound signals are transmitted from all the activated elements at the same time, the ultrasound signal transmitted from a nearest element to the focal point may reach the focal point first, and the farther the distance from an element to the focal point is, the greater the delay in the arrival time for the ultrasound signal transmitted from the element at the focal point. Therefore, as shown in FIG. 2, time delay may be taken into account even from when a transmit signal is originated, the transmit signal being an electric signal to be converted to an ultrasound signal in an element. Beamforming may be performed by originating the transmit signal later for an element nearer to the focal point and earlier for an element farther from the focal point.

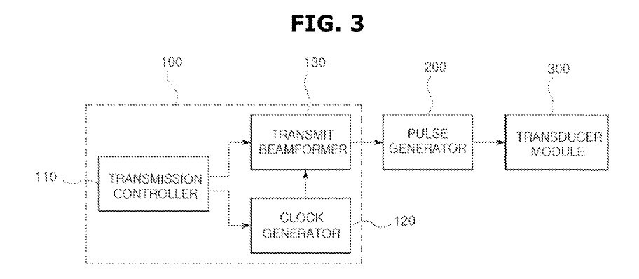

FIG. 3 is a control block diagram of a transmit beamforming apparatus, according to an exemplary embodiment.

Referring to FIG. 3, a transmit beamforming apparatus 100 may include a transmit beamformer 130 configured for performing beamforming on the transmit signal, a clock generator 120 configured for producing clock signals to apply a time delay to the transmit signal output from the transmit beamformer 130, and a transmission controller 110 configured for controlling the transmit beamformer 130 and the clock generator 120.

The transmit beamformer 130 may form a transmit signal pattern by applying a time delay to a transmit signal having a predetermined frequency or bandwidth. Information about the transmit signal may be provided from the transmission controller 110, pre-stored in the transmit beamformer 130, provided from another processor or controller in an ultrasonic probe in which the transmit beamformer 130 is included, or provided from a backend of the ultrasonic diagnosis apparatus that includes the transmit beamformer 130.

The clock generator 130 is a variable clock generator that is able to change a clock frequency. A clock signal produced by the clock generator 120 may be synchronized to a particular clock frequency and coded in a value of either zero (0) or one (1) based on the clock frequency. Once the clock generator 120 produces and inputs a transmit clock signal to the transmit beamformer 130, the transmit beamformer 130 may output a transmit signal per element according to the transmit clock signal.

The transmission controller 110 may calculate and dynamically set a clock frequency of the clock generator 120 based on particular parameters, which will be described in detail below.

A transmit signal output with a time delay from the transmit beamformer 130 may be input to the pulse generator 200, and the pulse generator 200 may generate a transmit pulse signal corresponding to the transmit signal and input the transmit pulse signal to the transducer module 300. The transducer module 300 may include a transducer array, which includes a plurality of transducer elements. The transducer array may be a 1D array or a 2D array.

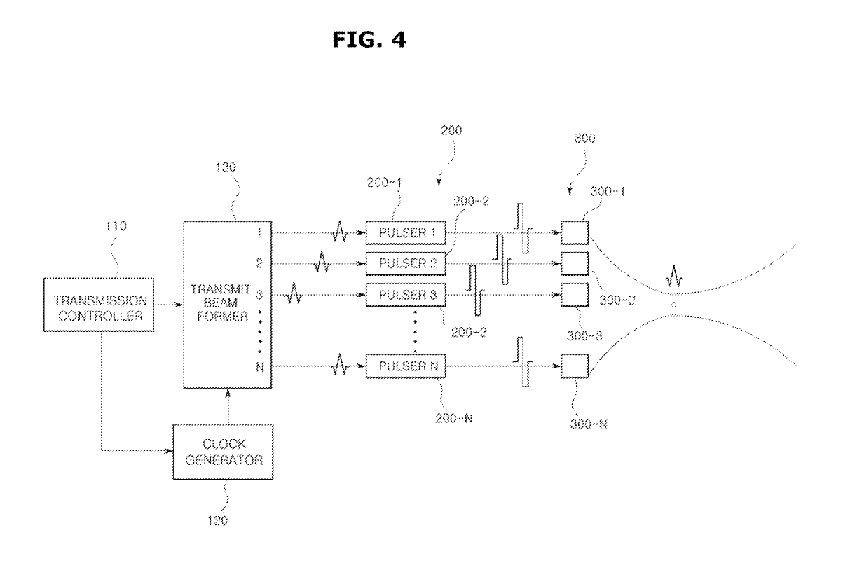

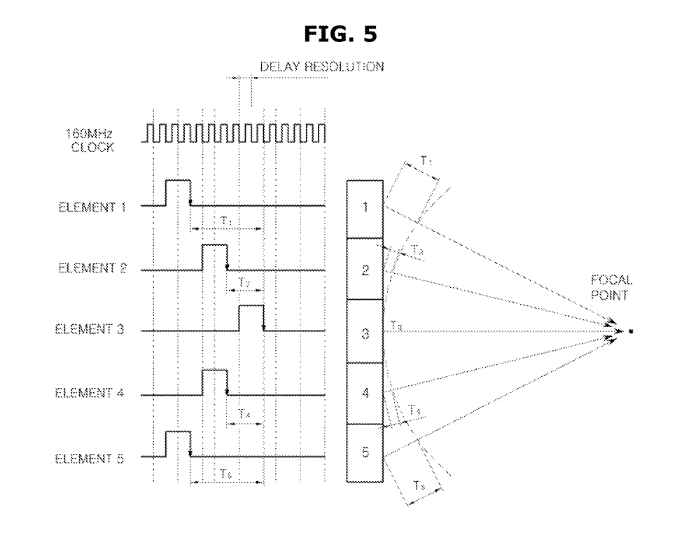

FIG. 4 schematically illustrates patterns of transmit signals output from the transmit beamformer and signal flows of the transmit signals until being focused on a focal point, and FIG. 5 illustrates a clock frequency of the clock generator and clock signals input to the transmit beamformer.

As shown in FIG. 4, assuming that the number of channels is N, the transmit beamformer 130 may output N transmit signals that have been subjected to transmit beamforming, and the pulse generator 200 may include N pulsers corresponding to the respective channels. In a case that a focal point is located in the center of the N channels, first element 300-1 and Nth element 300-N are farthest from the focal point. Accordingly, transmit signals on the first and Nth channels are output first from the transmit beamformer 130, and a transmit signal on a channel, which is closer to the center of the channels, is output at a later time.

The transmit signals which are time-delayed and output from the transmit beamformer 130 may be input to the pulsers 200-1 to 200-N, and the pulsers 200-1 to 200-N may generate high voltage transmit pulse signals and then input the generated signals to the elements 300-1 to 300-N. The elements 300-1 to 300-N may convert the transmit pulse signals to corresponding ultrasound signals in the order in which the transmit pulse signals are received, and irradiate the ultrasound signals toward the focal point. Accordingly, N ultrasound signals, which are in phase, may reach the focal point, which leads to improvement of the image resolution and the signal-to-noise ratio.

On the assumption that N=5 in the exemplary embodiment illustrated in FIG. 4, FIG. 5 will now be described. As shown in FIG. 5, assuming that a focal point is nearest to element 3 among five elements, transmit signals may be input to element 1 and element 5 first, to element 2 and element 4 next, and to element 3 last. With respect to element 3, element 1 may receive the transmit signal earlier by a time T₁, element 2 may receive the transmit signal earlier by a time T₂, element 4 may receive the transmit signal earlier by a time T₄, and element 5 may receive the transmit signal earlier by a time T₅, where T₁, T₂, T₄, and T₅ are delay times for the respective elements. T₁ and T₅ may be equal to each other, and/or T₂ and T₄ may be equal to each other. If the clock generator 120 is synchronized to a 160-MHz clock as shown in FIG. 5, the clock frequency of the clock generator 120 is 160 MHz, meaning that the clock generator 120 may output a signal once per 6.25 ns.

When the clock generator 120 inputs the transmit clock signal to the transmit beamformer 130, the transmission controller 110 may control the timing of the transmit clock signal by accounting for a transmission delay time per element. For this, the transmission controller 110 may calculate a transmission delay time for each element, and control the clock generator 120 to output the transmit clock signal in the transmission delay time per element.

In the exemplary embodiment illustrated in FIG. 5, the clock generator 120 may have a 160 MHz clock frequency and output a clock signal once per 6.25 ns. Accordingly, if T₁, T₂, T₄, and T₅ are multiples or divisors of 6.25 ns, the transmit clock signal may be output exactly in the calculated delay time, but otherwise, delay errors may occur.

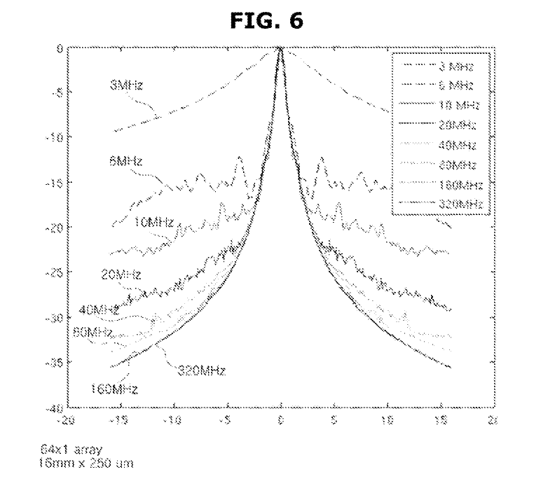

The influence of the clock frequency of the clock generator on the delay error will be described with reference to a graph of FIG. 6.

FIG. 6 is a graph representing beam profiles obtained at different clock frequencies, e.g., 3 MHz, 6 MHz, 10 MHz, 20 MHz, 40 MHz, 80 MHz, 160 MHz, and 320 MHz, with respect to a 64×1 1D transducer array.

The center of the horizontal axis represents the center of the transducer array, and the vertical axis represents directivity. Beamforming effect may be considered better when a difference between main and side lobes is greater. Referring to FIG. 6, when the clock frequency is 3 MHz, it is seen that beamforming is performed worst, because the difference between main and side lobes is smallest. As the clock frequency increases, the difference between main and side lobes increases, and accordingly, beamforming is seen to have been performed better.

Referring again to FIG. 5, it is seen that the greater the clock frequency of the clock generator 120, the less the delay error. Referring to FIG. 6, it may also be seen that as the clock frequency of the clock generator 120 increases, one may obtain better beamforming effect. In this aspect, the clock frequency of the clock generator 120 or the reciprocal thereof may be an index to indicate how often the clock generator 120 is able to output the clock signal or how fine the delay time may be controlled. Thus, the clock frequency of the clock generator 120 may be referred to as a delay sampling frequency or a delay frequency, and the reciprocal of the clock frequency may be referred to as delay resolution.

Although low delay resolution may reduce delay errors and improve a corresponding beamforming effect, low delay resolution also increases an amount of data to be processed in the transmit beamforming apparatus, thus making it difficult to simplify and miniaturize the hardware design. The transmit beamforming apparatus in accordance with an exemplary embodiment may dynamically set the delay frequency based on particular parameters, in order to restrict an amount of data to be processed in the transmit beamforming apparatus while obtaining an optimal beamforming effect.

Parameters that influence beamforming effects will now be described.

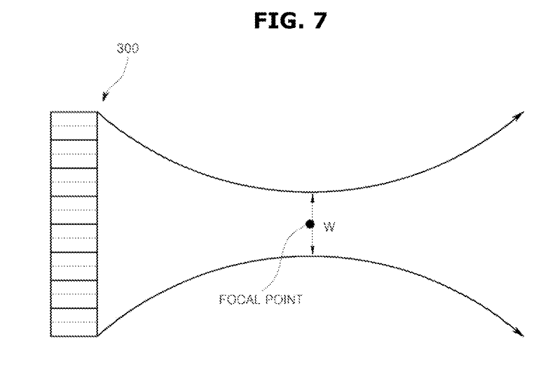

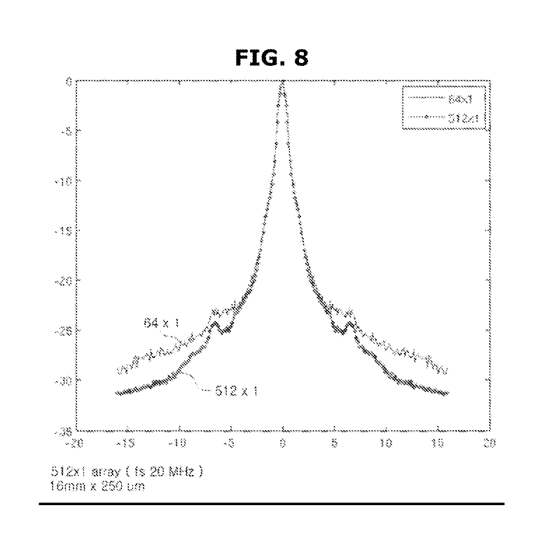

FIGS. 7 and 8 show a change in beamforming effect, which appears when delay information increases in 1D array transducers.

As shown in FIG. 7, if the number of elements is increased while an entire aperture size remains the same in a 1D transducer array, delay information increases, which leads to improvement of the beamforming effect.

Simulation results of this are represented in the graph shown in FIG. 8. In particular, respective beam profiles for 64×1 and 512×1 transducer arrays are obtained. In both cases, the delay frequency is 20 MHz.

As shown in FIG. 8, it is seen that, with the same delay frequency, the measurement of the difference between main and side lobes of the 512×1 transducer array is greater than that of the 64×1 transducer array. In this aspect, it is seen that the 512×1 transducer array yields a better beamforming effect. In particular, even if the same delay frequency is applied, a greater number of elements may lead to a better beamforming effect. Accordingly, the transmission controller 110 may account for the number of elements used for ultrasound imaging in setting the delay frequency.

Continuing to increase the number of elements while keeping the aperture size intact in the 1D transducer array might reduce the size and pitch of the elements too much, and thus, it may be difficult to implement such elements in a real-world practical application. Accordingly, an addition of an array in the 2D direction may be considered instead.

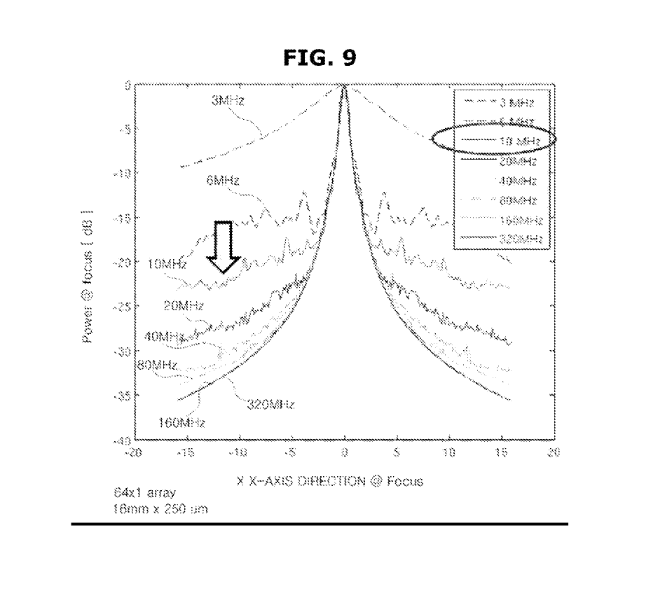

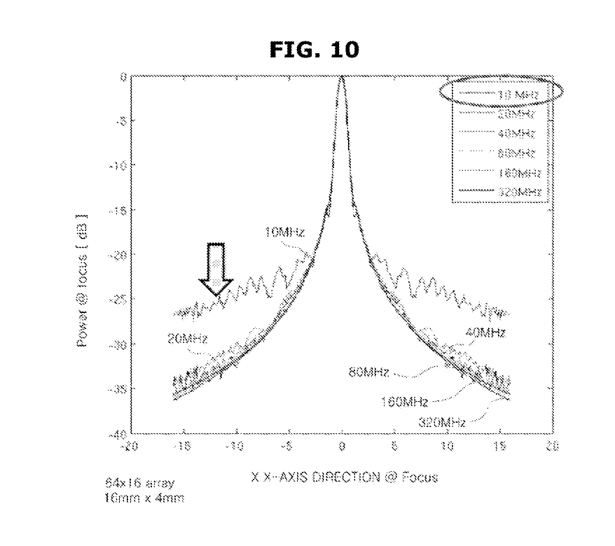

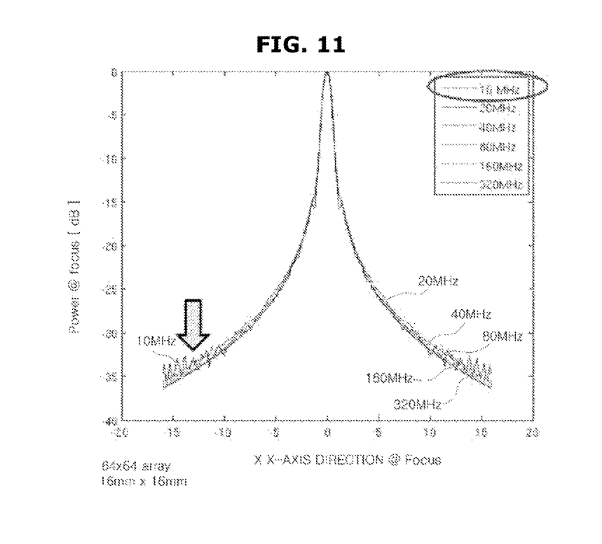

FIG. 9 is a graph representing beam profiles of a 64×1 1D transducer array, FIG. 10 is a graph representing beam profiles of a 64×16 2D transducer array, and FIG. 11 is a graph representing beam profiles of a 64×64 2D transducer array. Beam profiles shown in FIGS. 9, 10, and 11 are measured at 30 millimeters of focal depth.

In the graphs of FIGS. 9, 10, and 11, comparing beam profiles at 10 MHz of the delay frequency, it is seen that as the number of transducer elements increases in the 2D direction, the side lobes become smaller and as a result, the difference between the main and side lobes become greater, thereby improving the beamforming effect. In this aspect, even if the same delay frequency is employed, it is seen that a better beamforming effect may be obtained as the number of elements increases in the 2D direction. Accordingly, the transmission controller 110 may account for whether the elements employed in ultrasound imaging are arranged in a 2D array in setting the delay frequency.

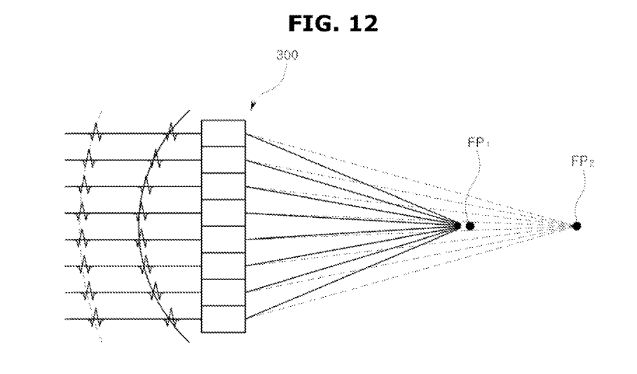

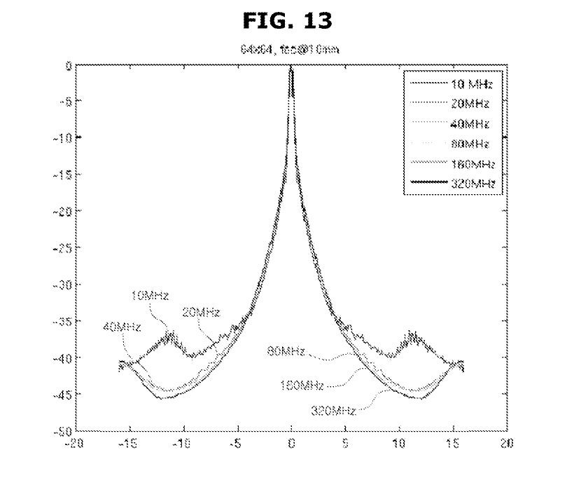

FIG. 12 shows a difference in delay time that varies as a function of focal depth, and FIG. 13 is a graph representing beam profiles of 64×64 2D transducer array at a 10 mm focal depth.

Referring to FIG. 12, the difference between a distance from a nearest element to the focal point and a distance from the farthest element to the focal point appears greater for a focal point FP₁ among two focal points FP₁ and FP₂, where FP₁ has a shorter focal depth than FP₂ does. In FIG. 12, signals shown on the left side of the transducer module 300 are transmit signals input to the transducer module 300, for which the respective elements may be linked into a delay profile. The delay profile shown in a solid line corresponds to FP₁, and the delay profile shown in a dotted line corresponds to FP₂. From the delay profiles, it is seen that the difference in delay time and maximum delay for each element appear greater for FP₁ than for FP₂.

When beam profiles of FIGS. 11 and 13 are compared with each other, both beam profiles are obtained with a 64×64 2D transducer array, and the beam profiles of FIG. 11 are obtained at 30 mm of focal depth, whereas the beam profiles of FIG. 13 are obtained at 10 mm of focal depth. It is seen that the beam profiles of FIG. 11 appear uniform across the entire delay frequency band to be measured, but the beam profiles of FIG. 13 have relatively large side lobes in some delay frequency bands, especially in 10 MHz of delay frequency band, thus yielding poor beamforming effect. Based on the graphs of FIGS. 11 and 13, with a 64×64 2D transducer array, if the focal depth is 30 mm, it is also possible to use 10 MHz of delay frequency, but if the focal depth is 10 mm, it may be preferable to use 20 MHz or more of delay frequency. In this aspect, assuming a same condition for the element, it is seen that better beamforming effect may be obtained at a longer focal depth even if a lower frequency is used. Accordingly the transmission controller 110 may account for the focal depth that is used for ultrasound imaging in setting the delay frequency.

Focal distance and aperture size and/or a designation of activated elements may depend on a location of the focal point, which will now be described in detail.

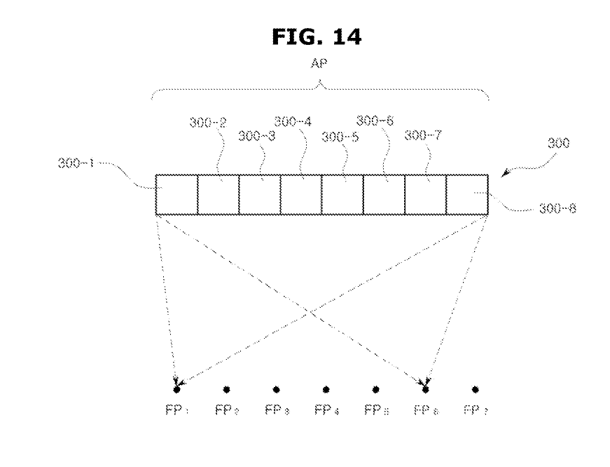

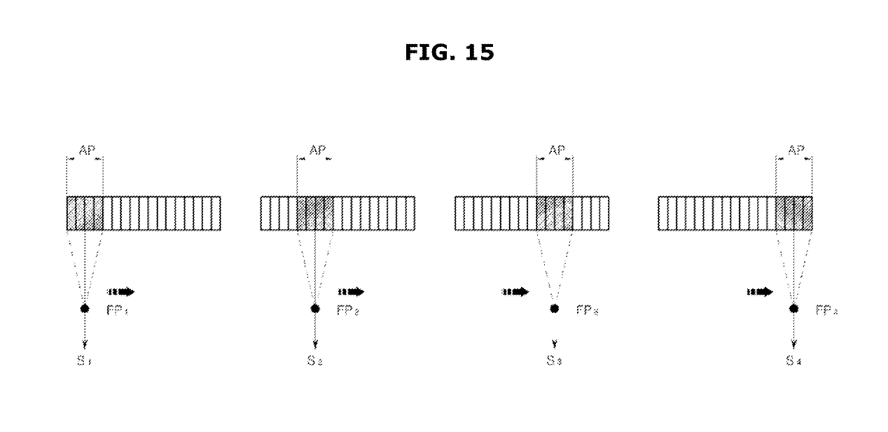

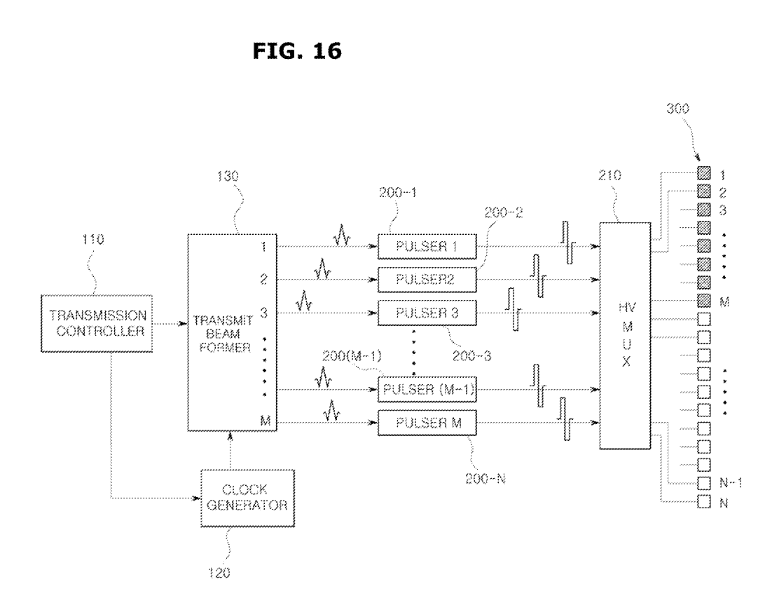

FIG. 14 shows a situation in which a designation of activated transducer elements remains the same even when a focal point is shifted laterally, FIG. 15 shows a situation in which a designation of activated transducer elements is changed when a focal point is shifted laterally, and FIG. 16 is a control block diagram of an ultrasonic probe to implement the situation illustrated in FIG. 15.

As discussed above in connection with FIG. 1, ultrasound signals may be transmitted for a plurality of scan lines in order to obtain a 2D cross-sectional image, and a focal point exists for each scan line. Accordingly, to obtain a 2D cross-sectional image, ultrasound signals may be transmitted by varying the location of the focal point.

As shown in FIG. 4, in a case of transmitting ultrasound signals with 8×1 1D array transducers toward seven focal points FP₁, FP₂, FP₃, FP₄, FP₅, FP₆, and FP₇ that lie in the lateral direction, the aperture size AP and locations of activated elements 300-1, 300-2, 300-3, 300-4, 300-5, 300-6, 300-7, 300-8 for all of the focal points may remain the same. Even in this case, however, the delay time applied to each respective element may vary. For example, a delay time for element 1 300-1 applied for focal point 1 FP1 may be different from a delay time for element 1 300-1 applied for focal point 7 FP7. Therefore, the transmission controller 110 may newly calculate and apply the respective delay time to be applied for each element, even if the delay frequency is equally applied for the respective focal points.

As shown in FIG. 15, it is also possible to change the location of the transducer elements to be activated while the aperture size AP is kept the same, as the focal point is shifted laterally.

For example, when four transducer elements may be activated for a single focal point, first to fourth elements may be activated to transmit ultrasound signals for focal point 1 FP₁; fifth to eighth elements may be activated to transmit ultrasound signals for focal point 2 FP₂; ninth to twelfth elements may be activated to transmit ultrasound signals for focal point 3 FP₃; and thirteenth to sixteenth elements may be activated to transmit ultrasound signals for focal point 4 FP₄.

For this, as shown in FIG. 16, a switch 210 may be included between the pulse generator 200 and the transducer module 300 and configured to selectively activate the transducer elements. For example, a high voltage (HV) multiplexer (MUX) 210 may be included to select elements to be activated depending on the focal point.

If the configuration of FIG. 16 is applied for the situation illustrated in FIG. 15, M=4 and N=16. As a result of receiving parameters that include focal depth, steering angle, the number of elements to be activated, aperture size, etc., the transmission controller 110 may be configured to calculate a delay frequency and delay time for each element based on the parameters, and to control the clock generator 120 according to the calculated delay frequency and delay time for each element.

In the case of transmitting ultrasound signals for focal point 1 FP₁, the transmit beamformer 130 may output transmit signals for first to fourth elements and the output timing of the transmit signals may be controlled by the clock generator 120 such that a delay time may be applied for each element. Delay errors that may occur at that time may depend on the delay frequency calculated by the transmission controller 110.

The output transmit signals are input to pulsers that correspond to four channels, e.g., pulser 1 200-1, pulser 2 200-2, pulser 3 200-3, and pulser 4 200-4, where M=4, and each pulser may output a transmit pulse signal by converting the input transmit signal to a pulse signal. The output four transmit pulse signals are input to the HV MUX 210, which may, in turn, output the transmit pulse signals to first to fourth elements among 16 transducer elements.

Upon reception of the transmit pulse signals, the first to fourth elements may convert the received signals to ultrasound signals and then transmit the converted signals to focal point 1, FP₁. As discussed above, since the transmission controller 110 calculates a delay time and the clock generator 120 controls the timing of outputting the transmit signal from the transmit beamformer 130 based on the calculated delay time, the ultrasound signals output from the four elements may reach focal point 1, i.e., FP₁, at the same time.

In the case of transmitting ultrasound signals for focal point 2, FP₂, the transmit beamformer 130 may output transmit signals for fifth to eighth elements and the output timing of the transmit signals may be controlled by the clock generator 120 such that a delay time may be applied for each element. Delay errors that may occur at that time may depend on the delay frequency calculated by the transmission controller 110, and the delay frequency for FP₂ may or may not be equal to the delay frequency for FP₁.

The output transmit signals are input to pulsers that correspond to four channels, e.g., pulser 1 200-1, pulser 2 200-2, pulser 3 200-3, and pulser 4 200-4, where M=4, and each pulser may output a transmit pulse signal by converting the input transmit signal to a pulse signal. The output four transmit pulse signals are input to the HV MUX 210, which may, in turn, output the transmit pulse signals to fifth to eighth elements among 16 transducer elements.

Upon reception of the transmit pulse signals, the fifth to eighth elements may convert the received signals to ultrasound signals and then transmit the converted signals toward focal point 2, i.e., FP₂. As discussed above, since the transmission controller 110 calculates a delay time and the clock generator 120 controls the timing of outputting the transmit signal from the transmit beamformer 130 based on the calculated delay time, the ultrasound signals output from the four elements may reach focal point 2, FP₂, at the same time.

In the case of transmitting ultrasound signals for focal point 3, FP₃, the transmit beamformer 130 may output transmit signals for ninth to twelfth elements and the output timing of the transmit signals may be controlled by the clock generator 120 such that a delay time may be applied for each element. Delay errors that may occur at that time may depend on the delay frequency calculated by the transmission controller 110, and the delay frequency for FP₃ may or may not be equal to the delay frequency for FP₁ or FP₂.

The output transmit signals are input to pulsers that correspond to four channels, e.g., pulser 1 200-1, pulser 2 200-2, pulser 3 200-3, and pulser 4 200-4, where M=4, and each pulser may output a transmit pulse signal by converting the input transmit signal to a pulse signal. The output four transmit pulse signals are input to the HV MUX 210, which may, in turn, output the transmit pulse signals to ninth to twelfth elements among 16 transducer elements.

Upon reception of the transmit pulse signals, the eighth to twelfth elements may convert the received signals to ultrasound signals and then transmit the converted signals toward focal point 3, FP₃. As discussed above, since the transmission controller 110 calculates a delay time and the clock generator 120 controls the timing of outputting the transmit signal from the transmit beamformer 130 based on the calculated delay time, the ultrasound signals output from the four elements may reach focal point 3, FP₃, at the same time.

In the case of transmitting ultrasound signals for focal point 4, FP₄, the transmit beamformer 130 may output transmit signals for thirteenth to sixteenth elements and the output timing of the transmit signals may be controlled by the clock generator 120 such that a delay time may be applied for each element. Delay errors that may occur at that time may depend on the delay frequency calculated by the transmission controller 110, and the delay frequency for FP₄ may or may not be equal to the delay frequency for FP₁, FP₂, or FP₃.

The output transmit signals are input to pulsers that correspond to four channels, e.g., pulser 1 200-1, pulser 2 200-2, pulser 3 200-3, and pulser 4 200-4, where M=4, and each pulser may output a transmit pulse signal by converting the input transmit signal to a pulse signal. The output four transmit pulse signals are input to the HV MUX 210, which may, in turn, output the transmit pulse signals to thirteenth to sixteenth elements among 16 transducer elements.

Upon reception of the transmit pulse signals, the thirteenth to sixteenth elements may convert the received signals to ultrasound signals and then transmit the converted signals toward focal point 4, FP₄. As discussed above, since the transmission controller 110 calculates a delay time and the clock generator 120 controls the timing of outputting the transmit signal from the transmit beamformer 130 based on the calculated delay time, the ultrasound signals output from the four elements may reach focal point 4, FP₄, at the same time.

Although it is assumed in FIG. 15 that the number of elements to be activated and the aperture size remain the same even if the focal point is shifted laterally, this is merely an example, and in an alternative exemplary embodiment, the aperture size may vary when the focal point is shifted depending on some imaging-related parameters or other conditions applied to the ultrasonic imaging.

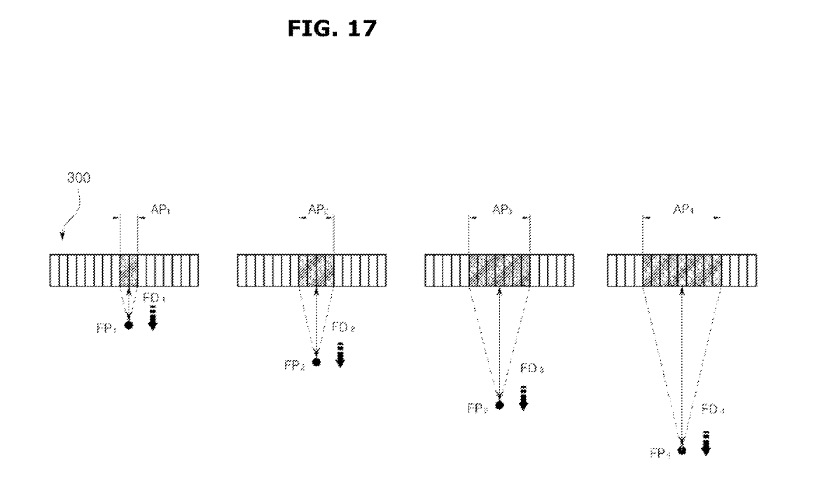

FIG. 17 shows changes in aperture size which correspond to the focal point being shifted in the axial direction.

In FIGS. 11, 12, and 13, a situation in which the aperture size AP remains the same even if the focal depth varies by axial movements of the focal point was described as an example. However, in some ultrasonic imaging situations, a different aperture size AP may be applied based on the focal depth by taking into account F numbers. This is called dynamic aperture, where the aperture does not refer to the entire size of the transducer module equipped in the ultrasonic probe but instead refers to a size defined by the number of elements to be activated depending on the focal point and the pitch.

F number is the ratio of the focal depth to the aperture size, and the smaller the F number, the brighter the image. Accordingly, an ultrasound image with a desired brightness may be obtained by properly controlling the F number, e.g., to be 0.5. The value of the F number may be applied for general ultrasonic imaging, but is not limited thereto, and may be set to any proper value other than 0.5.

In a case the F number is set to a certain value, the aperture size increases as the focal depth increases, and the aperture size decreases as the focal depth decreases. Thus, as shown in FIG. 17, as the focal point is shifted in the axial direction, e.g., FP₁→FP₂→FP₃→FP₄, focal depth increases, e.g., FD₁→FD₂→FD₃→FD₄ and the corresponding aperture size increases, e.g., AP₁→AP₂→AP₃→AP₄.

Based on what are described about the parameters that influence the delay frequency, an operation of the transmission controller 110 calculating the delay frequency will now be described.

First, the transmission controller 110 may calculate a respective delay time for each element based on certain parameters, such as the focal depth of the transducer module 300, the steering angle, the aperture size, the number of transducer elements to be activated, etc.

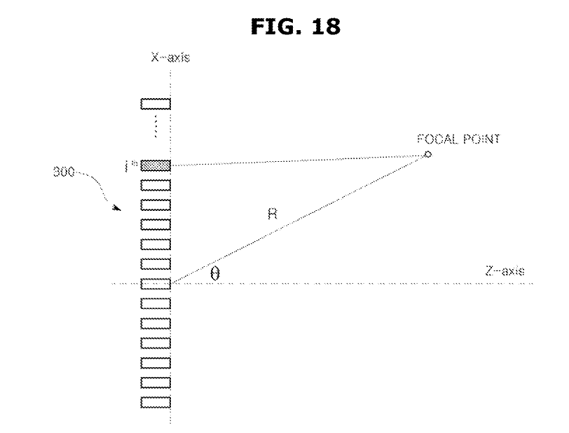

FIG. 18 shows a diagram which illustrates how a transmission controller calculates a delay time for a transducer element.

Assuming that the focal point is located as shown in FIG. 18, the steering angle is θ, the distance from the center of elements to be activated in the transducer module 300 to the focal point is R, and the distance in the X direction from the center of the elements to be activated to an i^{th} element for which delay time is to be calculated is xᵢ, a transmission delay time Tₜ may be calculated by using Equation (1) as follows:

$${T}_{t} = - \frac{R}{C} \sqrt{\frac{{x}_{i}^{2}}{{R}^{2}} - \frac{2 {x}_{i}}{R} sin θ} \qquad ( 1 )$$

where c represents the speed of sound.

If the transducer module 300 does not conduct steering, θ may be set to zero, i.e., θ=0. In this aspect, a respective delay time for each element may be calculated by setting the variables of Equation 1 to suit the imaging environment. Delay times may be calculated as necessary or calculated in advance and stored in the form of a table.

The transmission controller 110 may control the clock generator 120 to output transmit signals with the respective delay time for each element. At this time, dynamically setting the delay frequency of the clock generator 120 may help to minimize an amount of data to be processed, and thus yield the best beamforming effect. Specifically, the transmission controller 110 may calculate the delay frequency based on one or more of the parameters including focal depth, steering angle, the number of transducer element to be activated, aperture size, properties of transmit signals, whether the transducer module 300 has a 2D array, etc.

It is seen from FIGS. 7 and 8 that an improved beamforming effect is obtained with a greater number of elements in the same aperture of an 1D transducer array even if the same delay frequency is applied, and from FIGS. 9, 10, and 11 that an improved beamforming effect is obtained based on an increasing number of transducer elements in the 2D direction even if the same delay frequency is applied.

Furthermore, it is seen from FIGS. 12 and 13 that an improved beamforming effect is obtained with increased focal depth with respect to the same aperture even if the same delay frequency is applied. In connection with FIGS. 14, 15, and 16, a situation in which the focal distance and the aperture size remain the same even if the focal point varies is described above, and in connection with FIG. 17, a situation in which the aperture size varies by the F number when the focal depth is changed by a shift of the focal point in the axial direction is described above. Accordingly, the transmission controller 110 may calculate the delay frequency for each focal point. In calculating the delay frequency, the transmission controller 110 may account for one or more of the parameters including whether the transducer module 300 includes a 2D transducer array, the focal depth, the aperture size, the steering angle, the number of elements to be activated, etc. Specifically, the transmission controller 110 may calculate a lower delay frequency with the transducer module 300 having a 2D transducer array than with the transducer module 300 having an 1D transducer array (on condition of having the same number of elements), with a longer focal depth than with a shorter focal depth, with a greater aperture size than with a smaller aperture size, or with a larger number of elements than with a smaller number of elements even when the aperture size remains the same.

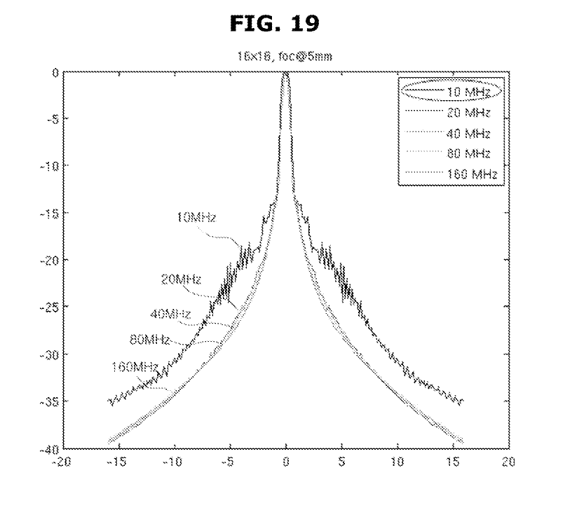

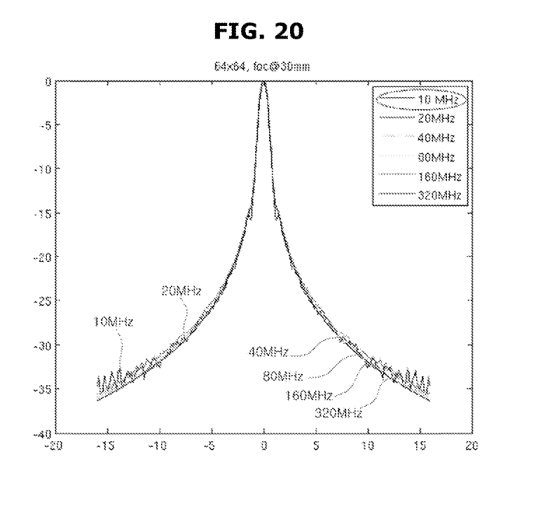

FIG. 19 is a graph that represents beam profiles with a focal distance of 5 mm and 16×16 2D array elements being activated, and FIG. 20 is a graph that represents beam profiles with a focal distance of 30 mm and 16×16 2D array elements being activated.

Referring to FIG. 19, with the focal distance of 5 mm and the 16×16 2D array elements being activated, an improved beamforming effect may be obtained provided that the delay frequency is 20 MHz or more. Accordingly, the transmission controller 110 may set the clock frequency of the clock generator 120 to 20 MHz.

Referring to FIG. 20, with the focal distance of 30 mm and the 16×16 2D array elements being activated, an improved beamforming effect may be obtained provided that the delay frequency is 10 MHz or more. Accordingly, the transmission controller 110 may set the delay frequency to 10 MHz.

A delay time to be applied to the transmit beamformer 130 may depend on the delay frequency. For example, if the delay frequency is 10 MHz, the delay resolution is 1/10 MHz=0.1 μs and thus, the transmit clock signal may be output once per 0.1 μs. This makes it difficult to apply an exact delay time for an element with 0.13 μs of delay time, so a new delay time may be calculated by accounting for the delay resolution.

Therefore, the transmission controller 110 may recalculate a respective delay time for each element based on the set delay frequency by applying Equation (2) as follows:

T_{d2}=Round[T_{d1}/T_{dr}]×T_{dr} (2)

where T_{d2} represents a second delay time recalculated based on the delay frequency, T_{d1} represents a first delay time (Tₜ as described above) calculated without regard to the delay frequency, and T_{dr} represents a delay resolution (i.e., 1/delay frequency). Round[*] represents a round function which rounds off a value in [*].

Based on an application of what are illustrated above in Equation 2, the first delay time Td1 is 0.13 μs and the delay resolution T_{dr} is 0.1 μs. As a result of Equation 2, the second delay time T_{d2} is 0.1 μs, and accordingly, the transmission controller 110 may control the transmit clock signal output from the clock generator 120 to be delayed as much as 0.1 μs for the corresponding element.

Exemplary embodiments of the transmit beamforming apparatus 100 that dynamically set a delay frequency have thus far been described. Since the concept about the time delay may also be equally applied when ultrasound echo signals are received, a receive beamforming apparatus in accordance with an exemplary embodiment may obtain an improved beamforming effect by dynamically setting the delay frequency, and may minimize an amount of data to be processed. Exemplary embodiments of a receive beamforming apparatus 400 that dynamically set a delay frequency will now be described in detail.

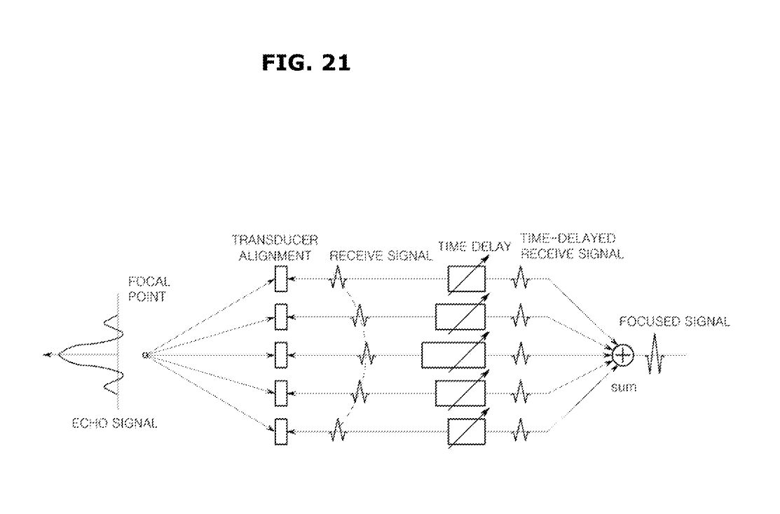

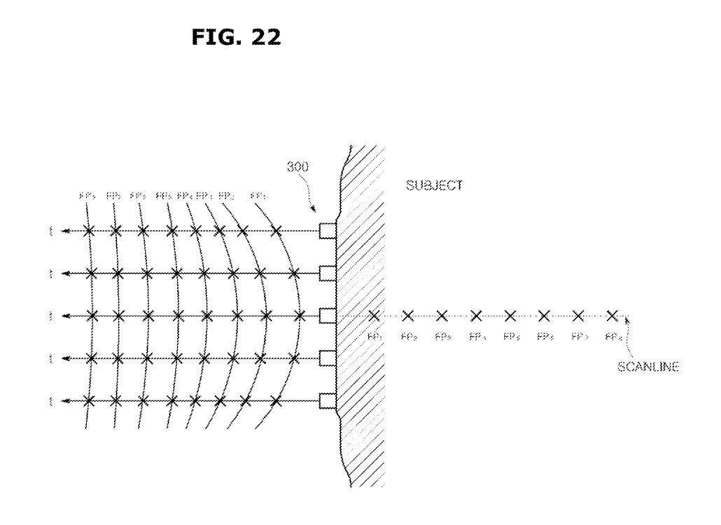

FIG. 21 shows a delay of receive signals to be applied in receive beamforming, and FIG. 22 shows dynamic reception focusing to be applied in receive beamforming.

As discussed above in connection with FIG. 2, when ultrasound signals in phase reach a focal point by performing transmit beamforming, echo signals are produced at the focal point and return to the transducer module 300. Similar to the case of transmitting ultrasound signals to the focal point, the respective distances to the focal point from the respective transducer elements are different, so the respective times for the ultrasound echo signals to reach their respective transducer elements is also different. Specifically, the ultrasound echo signal reaches a nearest element to the focal point first, and a farthest element from the focal point last. Exact location information is hardly obtained only with a single signal received by each element. Therefore, similar to transmit beamforming, receive beamforming entails applying appropriate delay times to respective receive signals that arrive at their respective elements at different points in time in order to combine them at the same time, thus improving the signal-to-noise ratio.

The concept of delay time and beamforming is also employed in receive beamforming, similarly as in transmit beamforming. However, receive beamforming may be performed for multiple focal points that lie on every scan line, whereas transmit beamforming is performed for a single focal point per scan line. This is because, even if ultrasound signals are irradiated to a single focal point, ultrasound echo signals are produced and received from multiple focal points that exist on the scan line. The points at which the ultrasound echo signals are produced is also called imaging points or focal points. In the following exemplary embodiments, not only a point from which the focused ultrasound signals are transmitted but also a point at which an ultrasound echo signal is produced from the focused ultrasound signal is referred to as a focal point, because the latter point is also used in receive focusing.

For example, as shown in FIG. 22, in a case in which ultrasound echo signals are produced at 8 focal points FP₁, FP₂, FP₃, FP₄, FP₅, FP₆, F₇, and FP₈ that exist on a scan line, an ultrasound signal produced from a nearest focal point to the transducer module 300, i.e., focal point 1, FP₁, reaches the transducer module 300 first, to build a delay profile with the biggest time difference per element. Subsequently, the ultrasound echo signals produced at focal points 2 to 8, FP₂ to FP₈, sequentially reach the transducer module 300, in which case the ultrasound echo signal produced at focal point 8, FP₈, that is farthest from the transducer module 300, builds a delay profile with the smallest time difference per element.

The receive beamforming apparatus 400, in accordance with an exemplary embodiment, may dynamically set the delay frequency to perform receive beamforming, by setting a respective delay frequency for every focal point on a scan line. Configuration and operation of the receive beamforming apparatus 400 will now be described in detail.

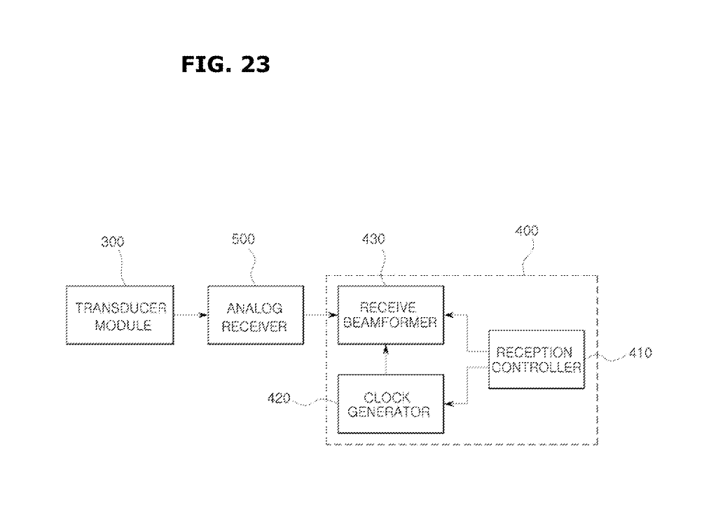

FIG. 23 is a control block diagram of the receive beamforming apparatus 400, according to an exemplary embodiment.

Referring to FIG. 23, the receive beamforming apparatus 400 may include a receive beamformer 430 which is configured for performing beamforming on the receive signal, a clock generator 420 which is configured for producing clocks to apply time delay to the receive signal output from the receive beamformer 430, and a reception controller 410 which is configured for controlling the receive beamformer 430 and the clock generator 420.

An ultrasound echo signal reflecting back from a focal point may be input to the transducer module 300, which may, in turn, convert the ultrasound echo signal into an analog electric signal.

The analog electric signal output from the transducer module 300 may be input to an analog receiver 500. The analog receiver 500 may amplify the analog electric signal and adjust a gain of the analog electric signal or compensate for attenuation due to the depth, before signal processing or signal delay processing is performed on the analog electric signal.

The receive beamformer 430 may be implemented as an analog beamformer to perform beamforming on the analog receive signal output from the analog receiver 500 by adding a delay time thereto before the analog electric signal is converted to a digital electric signal. Various methods may be applied by the receive beamformer 430 for a purpose of adding a delay time to the analog receive signal. For example, delay lines configured with inductor (L) and capacitor (C) elements may be used, and a delay time of each delay line may be controlled by the clock signal output by the clock generator 420. In another example, it is also possible to sample and hold the analog receive signals input from the analog receiver 500 in the input order so as to combine them at the same time, and the sampling timing may be controlled by the clock signal output by the clock generator 420. Alternatively, delay times are applied to the analog receive signals output from the analog receiver 500 by holding and then simultaneously outputting them. Any method for applying the delay time may be applied in various exemplary embodiments, provided that the method uses clock signals output from the clock generator 420 to control the delay time, and there are no limitations of arrangements or types of components used to implement the method.

A method which may be implemented by the receive beamformer 430 to sample and hold analog receive signals in the input order and to simultaneously output the signals will now be described as an example.

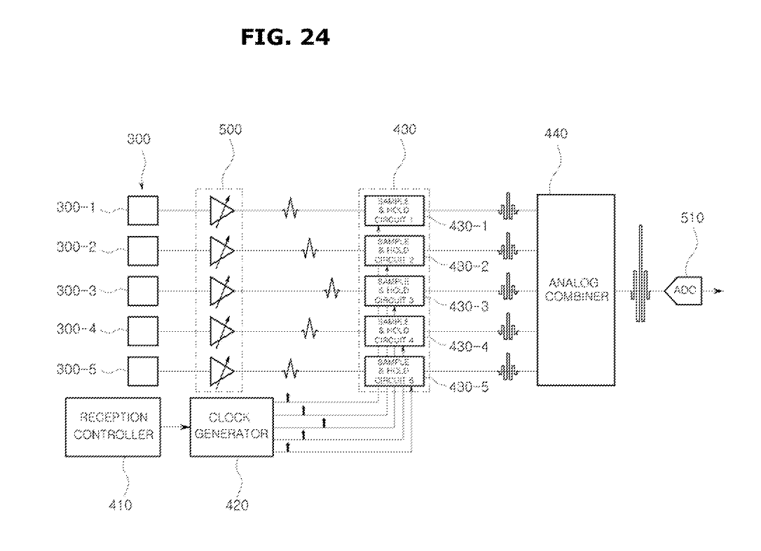

FIG. 24 shows patterns of receive clock signals input to a receive beamformer and receive signals output from the receive beamformer, in case of performing receive beamforming according to a sample and hold scheme.

As discussed above, the ultrasound echo signals reflecting back from the focal point to the transducer module 300 reach a nearest element to the focal point first and reach a farther element from the focal point at a later time.

The analog receiver 500 may perform amplification and gain adjustment on analog electric signals converted from the ultrasound echo signals as soon as they are output from the respective elements, and input the results to the receive beamformer 430. In a case that a third element 300-3 is the nearest element to the focal point from among five elements 300-1, 300-2, 300-3, 300-4, 300-5 included in the transducer module 300, the analog receive signal input to the receive beamformer 430 may have a pattern showing that a corresponding signal for the third element 300-3 is input first, as shown in FIG. 24.

The receive beamformer 430 may include as many sample and hold circuits 430-1, 430-2, 430-3, 430-4, 430-5 as the number of channels, each sample and hold circuit receiving an analog receive signal that corresponds to each respective element. As described above, since the ultrasound echo signal reaches the respective elements at different points in time, sampling timing in each sample and hold circuit is also different. Accordingly, the clock generator 420 may generate receive clock signals and input the generated receive clock signals to the sample and hold circuits in order to control the sampling timing in each sample and hold circuit.

As shown in FIG. 24, the receive clock signal may be input first to the third sample and hold circuit 430-3 to which the analog receive signal is input first, next to the second and fourth sample and hold circuits 430-2 and 430-4, and last to the first and fifth sample and hold circuits 430-1 and 430-5. The receive clock signal input to the sample and hold circuit is used to control the sampling timing, and may also be called a sample clock signal. Each sample and hold circuit may sample and hold the analog receive signal and then output it at the time when the receive clock signal is input. Output analog receive signals are combined by an analog combiner 440 and the result is sent to a back-end.

Although in the exemplary embodiment of FIG. 24, an analog-to-digital converter is not included in the receive beamforming apparatus 400 and the analog receive signal is sent to the back-end, exemplary embodiments of the receive beamforming apparatus 400 are not limited thereto and it is also possible for the receive beamforming apparatus 400 to have an analog-to-digital converter to send a digital receive signal to the back-end.

Similar to the clock generator 120 of the transmit beamforming apparatus 100, the clock generator 420 of the receive beamforming apparatus 100 may also be implemented as a variable clock generator that is able to generate clock signals with variable frequency. A clock signal produced by the clock generator 420 may be synchronized to a certain clock frequency and coded in value 0 or 1 based on the clock frequency. Since a delay time may be applied to the analog receive signal according to the receive clock signal input to the receive beamformer 430 from the clock generator 420, the clock frequency of the clock generator 420 may also be referred to as a delay frequency or as a delay sampling frequency, and its reciprocal corresponds to a delay resolution.

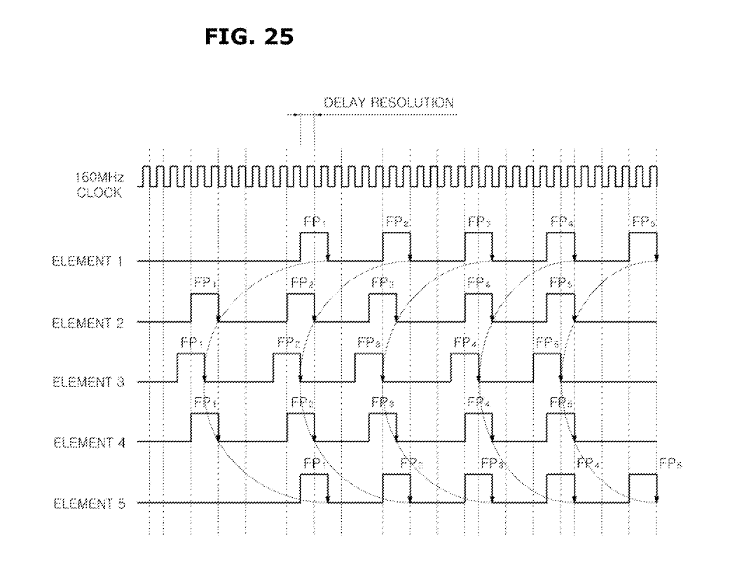

FIG. 25 is a timing diagram of receive clock signals for respective elements.

As shown in FIG. 22, the ultrasound echo signals are produced at a plurality of focal points that lie on a scan line and reach the transducer module 300 in the order from one produced at a nearest focal point to the transducer module 300 to one produced at a farthest focal point to the transducer module 300. In this example, it is shown that ultrasound echo signals produced at focal points 1 to 5, FP1 to FP5 reach the transducer module 300.

The clock generator 420 may generate a receive clock signal, as shown in FIG. 25, and input the generated receive clock signal to the respective sample and hold circuits. Sampling is performed at falling edges of the receive clock signal.

The transmission controller 410 may control the timing of the receive clock signal by accounting for a respective delay time for each element. For this, the transmission controller 410 may calculate a respective reception delay time for an analog receive signal output from each element, and control the clock generator 420 to output the receive clock signal in the reception delay time.

In this regard, a respective reception delay time may be calculated for every focal point on a single scan line, and the following Equation 3 may be used to calculate the reception delay time, which is represented by TR. Variables in Equation 3 are already described above in connection with Equation 1 and FIG. 18.

$${T}_{R} = \frac{R}{C} \sqrt{2 + \frac{{x}_{i}^{2}}{{R}^{2}} - \frac{2 {x}_{i}}{R} sin θ} \qquad ( 3 )$$

The respective reception delay times may be calculated by the reception controller 410 as necessary, or calculated in advance and stored in the form of a table.

In the example of FIG. 25, the clock frequency of the clock generator 420 is 160 MHz. In this case, the delay resolution is 6.25 ns, which means that a sample clock signal may be output once every 6.25 ns. Accordingly, if the respective reception delay times are multiples or divisors of 6.25 ns, the receive clock signal may be output exactly in the calculated reception delay time, but otherwise, delay errors may occur. How the clock frequency of the clock generator 420 influences the delay error is already described above with respect to an exemplary embodiment of the transmit beamforming apparatus 100.

Specifically, an improved beamforming effect may be obtained with a greater number of elements in the same aperture of an 1D transducer array or with an increase in the number of elements in the 2D direction, even if the same delay frequency is applied. Furthermore, beamforming effect may be further improved with increased focal depth for the same aperture even if the same delay frequency is applied. Even though the focal point varies by ultrasonic imaging methods, the focal distance and the aperture size may remain the same, or the aperture size may vary by the F number if the focal depth is changed by the shift of the focal point in the axial direction.

Accordingly, the reception controller 410 may calculate the respective delay frequency for each focal point. In calculating the delay frequency, the reception controller 410 may account for one or more of the parameters that include whether the transducer module 300 includes a 2D transducer array, the focal depth, the aperture size, the steering angle, the number of elements to be activated, etc.

The reception controller 410 may calculate a lower delay frequency with the transducer module 300 having a 2D transducer array than with the transducer module 300 having an 1D transducer array (on condition of having the same number of elements), with a longer focal depth than with a shorter focal depth, with a greater aperture size than with a smaller aperture size, or with a larger number of elements than with a smaller number of elements even when the aperture size remains the same.

Likewise, as described above with respect to an exemplary embodiment of the transmit beamforming apparatus 100, the delay time to be applied to the receive beamformer 430 may vary by the delay frequency. For example, if the delay frequency is 10 MHz, the corresponding delay resolution is 1/10 MHz=0.1 μs and thus, the receive clock signal may be output once per 0.1 μs. This makes it difficult to apply an exact delay time for an analog receive signal with 0.13 μs of delay time, so a new delay time may be calculated by accounting for the delay resolution. Accordingly, the reception controller 410 may recalculate a delay time for each analog receive signal based on a set delay frequency as in Equation 2, wherein Td1 of Equation 2 is substituted by the reception delay time TR calculated by Equation 3. The reception controller 410 may control the clock generator 420 to output a sample clock signal based on the recalculated delay time.

Similarly as described above with respect to the transmit beamforming apparatus 100, the receive beamforming apparatus 400 may also obtain an improved beamforming effect while restricting an amount of data to be processed, by dynamically setting the delay frequency.

The transmit beamforming apparatus 100 and the receive beamforming apparatus 400 may be included in the ultrasonic probe that corresponds to a front-end of the entire ultrasonic diagnosis device, or included in the main unit that corresponds to a back-end. Exemplary embodiments of the transmit beamforming apparatus 100 and the receive beamforming apparatus 400 are not limited thereto, so all or a part of components of the transmit beamforming apparatus 100 and the receive beamforming apparatus 400 may be included in any part of the front-end and back-end.

An exemplary embodiment of an ultrasonic probe that includes the transmit beamforming apparatus 100 and the receive beamforming apparatus 400 will now be described.

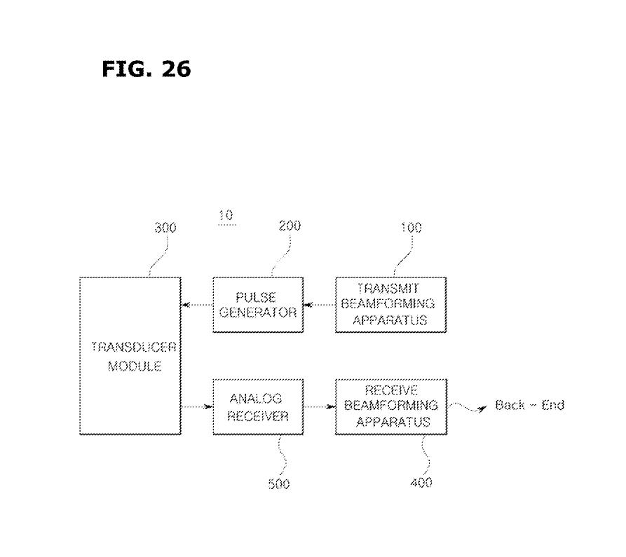

FIG. 26 is a control block diagram of an ultrasonic probe, according to an exemplary embodiment.

Referring to FIG. 26, an ultrasonic probe 10 may include the transmit beamforming apparatus 100 that enables ultrasound transmit signals to be focused, and the receive beamforming apparatus 400 that enables analog receive signals to be focused. The transmit beamforming apparatus 100 and the receive beamforming apparatus 400 are already described above, so the detailed description thereof will be omitted herein.

The ultrasonic probe 10 may further include the pulse generator 200 which is configured for generating a transmit pulse signal based on a transmit signal pattern formed to facilitate a focusing of the ultrasound transmit signals in the transmit beamforming apparatus 100, and the analog receiver 500 which is configured for performing amplification and gain adjustment on ultrasound echo signals received and converted by the transducer module 300 to electric signals.

The pulse generator 200 may include as many pulsers as the number of channels or transducer elements included in the transducer module 300, and for example, each pulser may generate voltage pulses of about −80 V to about +80 V or about 0 V to about 200 V and then input the generated voltage pulses to the respective elements included in the transducer module 300. The pulse generator 200 and the transducer module 300 may be connected to each other via a cable.

The transducer module 300 may be configured to convert the input transmit pulse signals to ultrasound signals and to irradiate the ultrasound signals toward a focal point. Since the transducer module 300 receives the transmit pulse signal that is time-delayed for each element, the ultrasound signals may leave the transducer module 300 in the order from a farthest element from the focal point and reach the focal point at the same time.

Once transmission of the ultrasound signals are completed for all the scan lines, the transducer module 300 switches modes to the reception mode from the transmission mode. For this, although not shown, the ultrasonic probe 10 may further include a transmission/reception switch to switch the transducer module 300 between transmission and reception modes.

Furthermore, as shown in FIG. 16, a switch which is similar to the HV MUX 210 may be further included between the pulse generator 200 and the transducer module 300 and may be configured to selectively activate transducer elements based on a location of the focal point.

As described above, ultrasound echo signals may be produced not only from the focal point of the transmit ultrasound signals, but also from a plurality of focal points that exist on a scan line. Accordingly, the transducer module 300 may receive and convert ultrasound echo signals produced at multiple focal points to electric signals, which are analog receive signals.

The analog receiver 500 may include a pre-amplifier which is configured for amplifying analog receive signals with very small amplitudes, and a low noise amplifier (LNA) may be employed as the pre-amplifier. Furthermore, a variable gain amplifier (VGA) may be included in order to control the gain of an input signal. In this case, time gain compensation (TGC) for compensating for the gain based on the focal point or the distance to the focal point may be employed, but exemplary embodiments of the ultrasonic probe 10 are not limited thereto.

The analog receive signal pre-processed by the analog receiver 500 is input to the receive beamforming apparatus 400, which in turn dynamically sets the delay frequency based on at least one of the parameters which include whether the transducer module 300 includes a 2D transducer array, the focal depth, the steering angle, the aperture size, the number of activated elements, etc., and then focuses the analog receive signal by applying a delay time to the analog receive signal based on the set delay frequency.

The focused analog receive signal is output from the ultrasonic probe 10 to a back-end. In the back-end, it is converted to a digital signal and undergoes various image processing operations for creating an ultrasound image. However, exemplary embodiments of the ultrasonic probe 10 are not limited thereto, and it is also possible to include an analog-to-digital converter in the ultrasonic probe 10 to convert the analog receive signal to a digital signal and send the digital signal to the back-end.

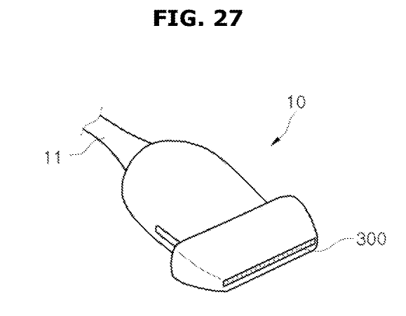

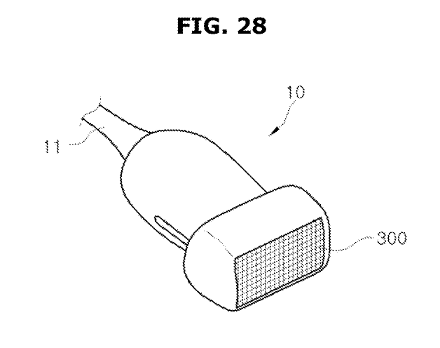

FIG. 27 is an exterior view of an ultrasonic probe that includes a 1D array of transducers, and FIG. 28 is an exterior view of an ultrasonic probe that includes a 2D array of transducers.

For example, the transducer module 300 may include a 1D array of transducers, as shown in FIG. 27. The 1D array of transducers are comprised of transducer elements, each of which may convert between ultrasound signals and electric signals. For this, the transducer elements may be implemented with any of magnetostrictive ultrasonic transducers that use magnetostrictive effects of a magnetic substance, piezoelectric ultrasonic transducers that use piezoelectric effects of a piezoelectric material, piezoelectric micromachined ultrasonic transducers (pMUTs), or the like, or may also be implemented with capacitive micromachined ultrasonic transducers (cMUTs) that transmit/receive ultrasounds using vibration of hundreds or thousands of micromachined thin films.

The ultrasonic probe 10 may have the transducer module 300 arranged in a linear array, as shown in FIG. 27, or in a convex array. The basic operating principle of the ultrasonic probe 10 is equally applied to both cases, but in case of the convex probe, ultrasound beams irradiated from the transducer module 300 are fan-shaped, and thus the resultant ultrasound image may also be fan-shaped.

In another example, the transducer module 300 may include a 2D array of transducers, as shown in FIG. 28. With the 2D array of transducers, the internal part of the subject may be imaged in three dimensions.

Each of the transducer elements included in the 2D transducer array is the same as that in the 1D transducer array, so the description of them will be omitted herein.

In the case the transducer module 300 includes the 2D array of transducers, an improved beamforming effect may be obtained than in the case of including the 1D array of transducers, even if the same delay frequency is applied. Therefore, in setting the delay frequency in the transmit beamforming apparatus 100 and the receive beamforming apparatus 400, a relatively lower delay frequency may be set, as compared to the case of including the 1D array of transducers.

The ultrasonic probe 10 may be connected to the main unit of the ultrasonic diagnosis device via a cable 11, which is configured for receiving various signals required to control the ultrasonic probe 10 and/or for sending analog or digital signals that correspond to ultrasonic echo signals received by the ultrasonic probe 10 to the main unit.

However, exemplary embodiments of the ultrasonic probe 10 are not limited thereto, and may be implemented with a wireless probe to exchange signals with the main unit over a network formed between the ultrasonic probe 10 and the main unit.

It is also possible that the clock generator 120 and transmission controller 110 of the transmit beamforming apparatus 100 are implemented with the same configuration as that of the clock generator 420 and reception controller 410 of the receive beamforming apparatus 400.

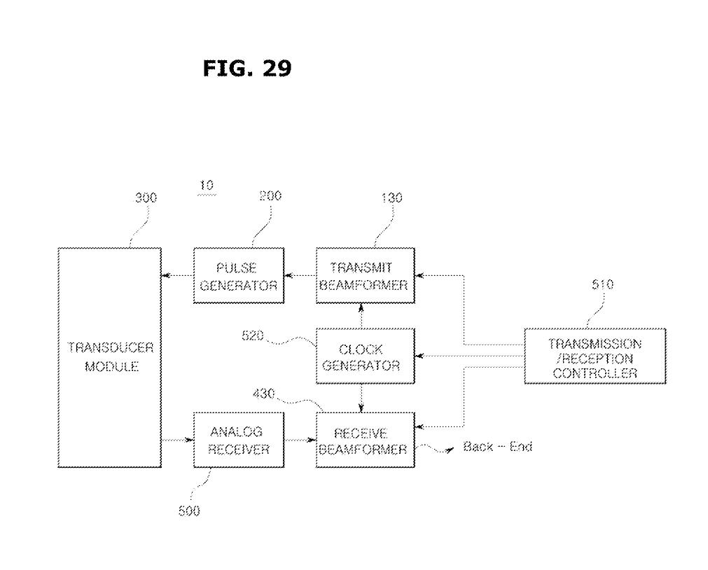

FIG. 29 is a control block diagram of transmit and receive beamforming apparatuses that share a clock generator and a controller.

Referring to FIG. 29, the transmit and receive beamforming apparatuses 100 and 400 are not implemented as separate modules in the ultrasonic probe 10. Rather, functions of the transmission controller 110 and the reception controller 410 may be performed by a controller 510, and functions of the clock generator 120 of the transmit beamforming apparatus 100 and the clock generator 120 of the receive beamforming apparatus 400 may be performed by a clock generator 520.

In addition to the clock generator and controller, other components may not be physically separated, but instead may share part or all of the physical configurations of the respective components, and a single physical unit may perform functions of a plurality of components.

An exemplary embodiment of an ultrasonic diagnostic apparatus that includes the transmit beamforming apparatus 100 and the receive beamforming apparatus 400 will now be described.

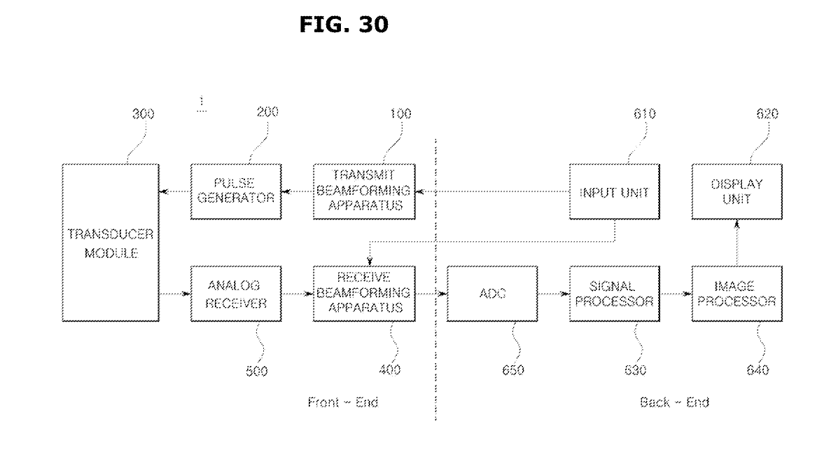

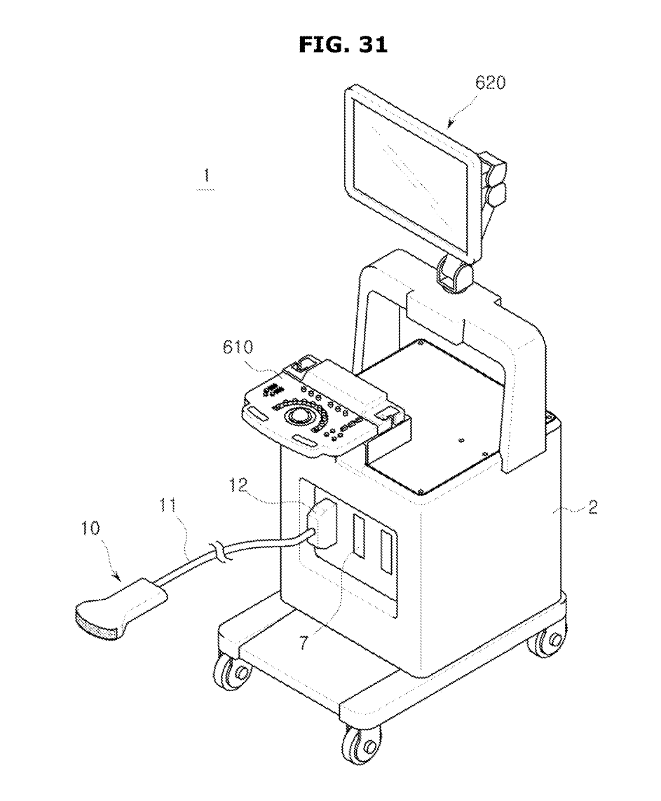

FIG. 30 is a control block diagram of an ultrasonic diagnostic apparatus, according to an exemplary embodiment, and FIG. 31 is an exterior view of an ultrasonic diagnostic apparatus, according to an exemplary embodiment. Referring to FIG. 30, an ultrasonic diagnostic apparatus 1 in accordance with an exemplary embodiment may include the transmit beamforming apparatus 100, the pulse generator 200, the transducer module 300, the receive beamforming apparatus 400, the analog receiver 500, an analog-to-digital converter 650, an input unit 610 and a display unit 620, which correspond to a user interface, a signal processor 630 which is configured for performing signal processing on a digital signal converted from a focused analog receive signal by the receive beamforming apparatus 400, and an image processor 640 which is configured for performing various types of conversion and image processing for image data to be displayed on the display unit 620.

For example, the signal processor 630 may be implemented with a digital signal processor (DSP), which is configured to form ultrasound image data by performing envelope detection to detect the amplitude of the ultrasound echo signals based on the focused digital receive signal. Specifically, the signal processor 630 may form ultrasound image data based on information about locations of the focal points that lie on each scan line and data obtained at each focal point. The ultrasound image data may include any of coordinates of each focal point in the XY coordinate system, information about an angle of each scan line against a vertical scan line, data obtained at each focal point, etc.

The image processor 640 may include a scan converter which is configured for performing scan conversion on the ultrasound image data to be displayed on the display unit 620. Furthermore, in order to display the ultrasound image on the display unit 620 in a mode desired by the user, various image processing operations may be performed on the scan-converted ultrasound image data. For example, the image processor 640 may create ultrasound images in many different modes, such as amplitude mode (A mode), brightness mode (B mode), motion mode (M mode), Doppler mode, etc., and display the images on the display unit 620. In the case the ultrasonic probe 10 includes the 2D array transducers or 1D array transducers that may be driven in the elevational direction, it is also possible to create a 3D ultrasound image by using a plurality of 2D cross-sectional images.

Referring to FIGS. 30 and 31, the ultrasonic probe 10 may be connected to a main unit 2 of the ultrasonic diagnostic apparatus 1. Once an analog receive signal is sent to the main unit 2 from the ultrasonic probe 10 via a cable 11, the analog-to-digital converter 650 may convert the analog receive signal to a digital receive signal, and the signal processor 630 and the image processor 640 may perform the aforementioned functions on the digital receive signal to create an ultrasound image.

One end of the cable 11 is connected to the ultrasonic probe 10, and the other end of the cable 11 is connected to a connector 12 that may be combined with or detached from a slot 7 of the main unit 2. With the cable 11, control commands or data may be exchanged between the main unit 2 and the ultrasonic probe 10. For example, when the user inputs information about the focal depth, the aperture size, the steering angle or the like, the information may be sent to the ultrasonic probe 10 via the cable 11 and used by the transmit beamforming apparatus 100 and the receive beamforming apparatus 400 to set the delay frequency. Alternatively, in the case the ultrasonic probe 10 is implemented with a wireless probe as described above, the ultrasonic probe 10 is connected to the main unit 2 not through the cable 11, but instead through a wireless network. Even in the case that the ultrasonic probe 10 is connected to the main unit 2 over the wireless network, control commands or data may be exchanged between the main unit 2 and the ultrasonic probe 10.

Exemplary embodiments of a transmit beamforming method and receive beamforming method will now be described.

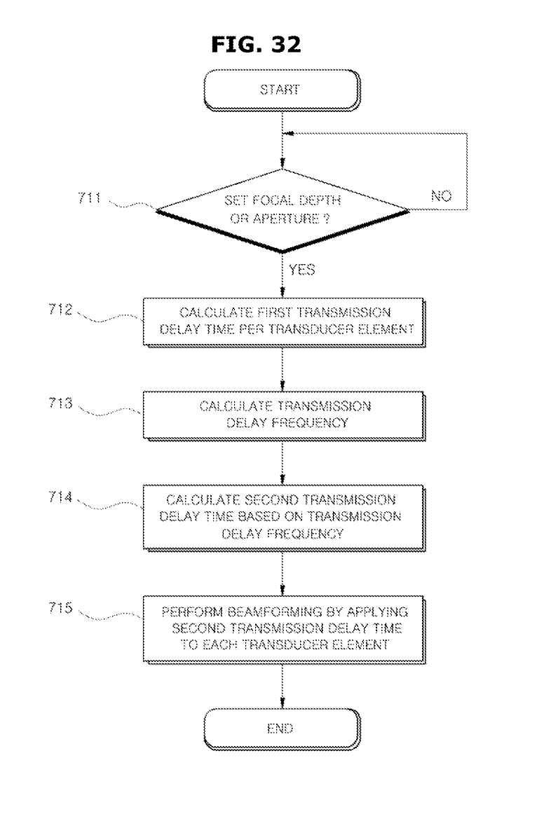

FIG. 32 is a flowchart illustrating a transmit beamforming method, according to an exemplary embodiment. The transmit beamforming method according to an exemplary embodiment may be employed by the transmit beamforming apparatus 100.

Referring to FIG. 32, once the focal depth or the aperture size is set in operation 711, first transmission delay times for the respective transducer elements used to transmit ultrasound signals to the focal point are calculated, in operation 712. In addition, in case that steering is conducted, the steering angle may also be set, and before the first transmission delay times are calculated, transducer elements to be activated may be set based on the focal depth, the aperture size, or the steering angle. The first transmission delay times may then be calculated for the transducer elements to be activated. Equation 1 may be used to calculate the first transmission delay time.

Transmission delay frequency is calculated by taking into account at least one of the parameters that include whether the transducer module 300 includes 2D array transducers, the focal depth, the aperture size, the number of elements to be activated, the steering angle, etc., in operation 713. Specifically, the transmission controller 110 may calculate a lower transmission delay frequency when the transducer module 300 has a 2D transducer array than when the transducer module 300 has a 1D transducer array, with a longer focal depth than with a shorter focal depth, with a greater aperture size than with a smaller aperture size, or with a larger number of elements than with a smaller number of elements even when the aperture size remains the same. Detailed description about the calculation of the delay frequency is the same as that described above with reference to FIGS. 1 to 20.

The delay frequency is the reciprocal of the delay resolution, so the delay time to be applied to a transmit signal may depend on the delay resolution. Accordingly, delay time per element may be recalculated based on the transmission delay frequency, which is called the second transmission delay time. The second transmission delay time may be calculated by substituting the first delay time and delay frequency in Equation 2, in operation 714.

Transmit beamforming is performed by applying the delay frequency and the second transmission delay time for each transducer element, in operation 715.

Once scanning is completed for one scan line or one focal point, another focal point starts to be scanned. As such, every time the focal point is shifted, operations 711, 712, 713, 714, and 715 are repeatedly performed in sequence, to create a 2D cross-sectional image.

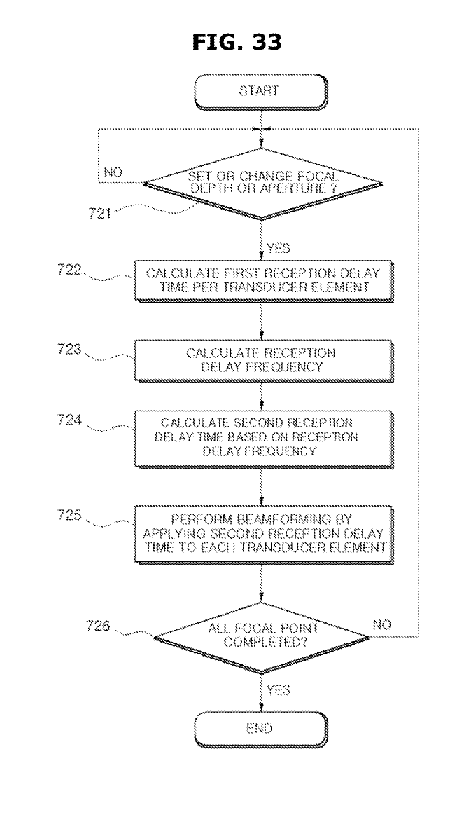

FIG. 33 is a flowchart illustrating a receive beamforming method, according to an exemplary embodiment. The receive beamforming method according to an exemplary embodiment may be employed by the receive beamforming apparatus 400.

Referring to FIG. 33, once the focal depth or the aperture size is set or changed in operation 721, a respective first reception delay time for each transducer element used for focusing of the receive signal received from the focal point is calculated, in operation 722.

If the location of the transducer element that is used to receive the ultrasound echo signal is shifted according to the location of the focal point, the location of an element to be activated may be set before the first reception delay time is calculated, and then the first reception delay time is calculated for the set element. Equation 3 may be used to calculate the first reception delay time.

A reception delay frequency is calculated by taking into account at least one of the parameters including whether the transducer module 300 includes 2D array transducers, the focal depth, the aperture size, the number of elements to be activated, the steering angle, etc., in operation 723. Specifically, the reception controller 410 may calculate a lower reception delay frequency when the transducer module 300 has a 2D transducer array than when the transducer module 300 has a 1D transducer array (on condition of having the same number of elements), with a longer focal depth than with a shorter focal depth, with a greater aperture size than with a smaller aperture size, or with a larger number of elements than with a smaller number of elements even when the aperture size remains the same. Detailed description about the calculation of the delay frequency is the same as that described above with reference to FIGS. 21 to 25.

The delay frequency is the reciprocal of the delay resolution, so the delay time to be applied to a receive signal may depend on the delay resolution. Accordingly, delay time per element may be recalculated based on the reception delay frequency, which is called second reception delay time. The second reception delay time may be calculated by substituting the first reception delay time and reception delay frequency in Equation 2, in operation 724.

Receive beamforming is performed by applying the reception delay frequency and the second reception delay time for each transducer element, in operation 725.

Once focused analog receive signals are output from the receive beamformer 430 as a result of receive beamforming, the main unit of the ultrasonic diagnostic apparatus 1, which corresponds to a back-end, may create an ultrasound image with the analog receive signal. In operation 726, a determination is made as to whether beamforming has been performed for all focal points. There are a number of focal points on a single scan line as shown in FIG. 22, and operations 721, 722, 723, 724, and 725 may be repeatedly performed in sequence until receive beamforming is completed for all the focal points. Once scanning is completed for one scan line, another scan line starts to be scanned. As such, every time the scan line is changed, operations 721 to 726 are repeatedly performed, to create a 2D cross-sectional image.

According to the aforementioned exemplary embodiments of the transmit beamforming apparatus, receive beamforming apparatus, ultrasonic probe and ultrasonic diagnostic apparatus having the same, transmit beamforming method and receive beamforming method, delay frequency may be dynamically set by taking into account the location of the focal point, the number of elements to be activated or their locations, signal properties, etc., thereby improving a beamforming effect while minimizing an amount of data to be processed in the apparatus.

With an analog beamformer employed in the apparatuses and methods, complexity of hardware structures may be avoided and total area of the ultrasonic probe may be reduced, thus achieving miniaturization of the ultrasonic probe.

The foregoing exemplary embodiments and advantages are merely exemplary and are not to be construed as limiting. The present teaching can be readily applied to other types of apparatuses. The description of the exemplary embodiments is intended to be illustrative, and not to limit the scope of the claims, and many alternatives, modifications, and variations will be apparent to those skilled in the art.

What is claimed is:

1. A transmit beamforming apparatus for transmitting ultrasound beams, the transmit beamforming apparatus comprising:
a transmit beamformer configured to form a transmit signal pattern by applying a delay time to a transmit signal that corresponds to at least one from among a plurality of ultrasonic transducer elements; and
a transmission controller configured to determine a delay frequency to be applied to the transmit signal in conjunction with the application of the delay time.

2. The transmit beamforming apparatus of claim 1, further comprising: a clock generator which is synchronized to the delay frequency and configured to generate a transmit clock signal which is usable for controlling an output timing of the transmit signal.

3. The transmit beamforming apparatus of claim 1, wherein the transmission controller is further configured to determine the delay frequency based on at least one from among whether the plurality of ultrasonic transducer elements is arranged in a two-dimensional (2D) array, a focal depth, a number of ultrasonic transducer elements to be activated, a steering angle, and an aperture size.

4. The transmit beamforming apparatus of claim 3, wherein the transmission controller is further configured to determine the delay frequency to vary inversely with respect to the focal depth.

5. The transmit beamforming apparatus of claim 3, wherein the transmission controller is further configured to determine the delay frequency to vary inversely with respect to the number of ultrasonic transducer elements to be activated.

6. The transmit beamforming apparatus of claim 3, wherein the transmission controller is further configured to determine the delay frequency to vary inversely with respect to the aperture size.

7. The transmit beamforming apparatus of claim 3, wherein the transmission controller is further configured to determine the delay frequency to be lower when the plurality of ultrasonic transducer elements is arranged in a 2D array than when the plurality of ultrasonic transducer elements is arranged in a one-dimensional (1D) array.

8. The transmit beamforming apparatus of claim 3, wherein the transmission controller is further configured to calculate a first delay time for the at least one from among the plurality of ultrasonic transducer elements based on at least one from among whether the plurality of ultrasonic transducer elements is arranged in a two-dimensional (2D) array, the focal depth, the number of ultrasonic transducer elements to be activated, the steering angle, and the aperture size.

9. The transmit beamforming apparatus of claim 8, wherein the transmission controller is further configured to calculate a second delay time for the at least one from among the plurality of ultrasonic transducer elements based on the determined delay frequency and the first delay time.

10. The transmit beamforming apparatus of claim 9, wherein the transmit beamformer is further configured to form the transmit signal pattern by applying the second delay time to the transmit signal that corresponds to the at least one from among the plurality of ultrasonic transducer elements.

11. A receive beamforming apparatus for focusing of analog receive signals, the receive beamforming apparatus comprising:
a receive beamformer configured to apply a delay time to analog receive signals converted from ultrasound echo signals received by at least one from among a plurality of ultrasonic transducer elements, and to focus the delayed analog receive signals; and
a reception controller configured to determine a delay frequency to be applied to the analog receive signals in conjunction with the application of the delay time.

12. The receive beamforming apparatus of claim 11, wherein the receive beamformer is further configured to hold the analog receive signals in order to apply the delay time to the analog receive signals.

13. The receive beamforming apparatus of claim 12, further comprising: a clock generator configured to generate a receive clock signal which is usable for controlling a holding time which corresponds to the holding the analog receive signals.

14. The receive beamforming apparatus of claim 13, wherein the clock generator is further configured to be synchronized to the delay frequency in order to generate the receive clock signal.

15. The receive beamforming apparatus of claim 14, wherein the receive beamformer is further configured to sample and hold the analog receive signals converted by the at least one from among the plurality of ultrasonic transducer elements based on the applied delay time, and to simultaneously output the sampled and held analog receive signals.

16. The receive beamforming apparatus of claim 11, wherein the reception controller is further configured to determine the delay frequency based on at least one from among whether the plurality of ultrasonic transducer elements is arranged in a two-dimensional (2D) array, a focal depth, a number of ultrasonic transducer elements to be activated, a steering angle, and an aperture size.

17. The receive beamforming apparatus of claim 16, wherein the reception controller is further configured to calculate a first delay time for the at least one from among the plurality of ultrasonic transducer elements based on at least one from among whether the plurality of ultrasonic transducer elements is arranged in a two-dimensional (2D) array, the focal depth, the number of ultrasonic transducer elements to be activated, the steering angle, and the aperture size, and to calculate a second delay time for the at least one from among the plurality of ultrasonic transducer elements based on the determined delay frequency and the first delay time.

18. The receive beamforming apparatus of claim 17, wherein the reception beamformer is further configured to apply the second delay time to the analog receive signals to be focused.

19. An ultrasonic probe comprising:
a plurality of ultrasonic transducer elements configured to transmit at least one ultrasound signal toward a focal point, to receive ultrasound echo signals which originate from the focal point, and to convert the received at least one ultrasound echo signal to at least one analog receive signal;
a transmit beamformer configured to delay a transmit signal input to at least one from among the plurality of ultrasonic transducer elements in order to focus the at least one ultrasound signal with respect to the focal point;
a receive beamformer configured to delay the at least one analog receive signal in order to focus the at least one analog receive signal; and
a controller configured to determine a delay frequency to be applied to at least one from among the transmit signal and the at least one analog receive signal.

20. The ultrasonic probe of claim 19, further comprising: a clock generator configured to generate a transmit clock signal which is usable for controlling an output timing of the transmit signal, and wherein the receive beamformer is further configured to hold the at least one analog receive signal in order to apply a delay time to the at least one analog receive signal.

21. A method for performing beamforming with respect to a transmit signal to be transmitted by at least one from among a plurality of ultrasonic transducer elements, the method comprising:
forming a transmit signal pattern by applying a delay time to the transmit signal; and
determining a delay sampling frequency to be applied to the transmit signal in conjunction with the applying the delay time,
wherein the determining the delay sampling frequency comprises varying the delay sampling frequency based on at least one characteristic which relates to the at least one from among the plurality of ultrasonic transducer elements.

22. The method of claim 21, wherein the at least one characteristic includes whether the plurality of ultrasonic transducer elements are arranged in a two-dimensional (2D) array.

23. The method of claim 21, wherein the at least one characteristic includes a focal depth of the at least one from among the plurality of ultrasonic transducer elements.

24. The method of claim 21, wherein the at least one characteristic includes a number of ultrasonic transducer elements to be activated from among the plurality of ultrasonic transducer elements.

25. The method of claim 21, wherein the at least one characteristic includes a steering angle of the at least one from among the plurality of ultrasonic transducer elements.

26. The method of claim 21, wherein the at least one characteristic includes an aperture size of the at least one from among the plurality of ultrasonic transducer elements.

27. A method for performing beamforming with respect to at least one analog receive signal to be received from at least one from among a plurality of ultrasonic transducer elements, the method comprising:
applying a delay time to the at least one analog receive signal which is converted from at least one ultrasound echo signal received by the at least one from among the plurality of ultrasonic transducer elements; and
determining a delay sampling frequency to be applied to the at least one analog receive signal in conjunction with the applying the delay time,
wherein the determining the delay sampling frequency comprises varying the delay sampling frequency based on at least one characteristic which relates to the at least one from among the plurality of ultrasonic transducer elements.

28. The method of claim 27, wherein the at least one characteristic includes whether the plurality of ultrasonic transducer elements are arranged in a two-dimensional (2D) array.

29. The method of claim 27, wherein the at least one characteristic includes a focal depth of the at least one from among the plurality of ultrasonic transducer elements.

30. The method of claim 27, wherein the at least one characteristic includes a number of ultrasonic transducer elements to be activated from among the plurality of ultrasonic transducer elements.

31. The method of claim 27, wherein the at least one characteristic includes a steering angle of the at least one from among the plurality of ultrasonic transducer elements.

32. The method of claim 27, wherein the at least one characteristic includes an aperture size of the at least one from among the plurality of ultrasonic transducer elements.
